(19) United States
(12) Patent Application Publication
(10) Pub. No.: US 2007/0142319 A1
(43) Pub. Date: Jun. 21, 2007

(54) Anxiolytic treatment by inhibition of a polysialyltransferase

(75) Inventors: Jamey Marth, San Diego, CA (US); Minoru Fukuda, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Burnham Institute, Le Jolla, CA (US); The Regenta of the University of California Office of Technology Transfer, Oakland, CA (US)

(21) Appl. No.: 10/589,835

(22) PCT Filed: Feb. 17, 2005
(86) PCT No.: PCT/US05/05328

§ 371 Date: Aug. 17, 2006

Related U.S. Application Data
(60) Provisional application No. 60/546,347, filed on Feb. 20, 2004.

(51) Int. Cl. A61K 31/715

(52) U.S. Cl. 514/54

(57) ABSTRACT

The invention provides methods and compositions for treating anxiety and fear disorders through inhibition of α-2,8-sialyltransferase activity.

### CROSS-REFERENCES TO RELATED APPLICATIONS

This application claims the benefit of U.S. Provisional Application No. 60/546,347, filed on Feb. 20, 2004, the entire disclosure of which is hereby incorporated by reference.

### STATEMENT AS TO RIGHTS TO INVENTIONS MADE UNDER FEDERALLY SPONSORED RESEARCH AND DEVELOPMENT

This invention was made with government support under grant numbers DK48247, HL57345, GM62116 awarded by the National Institutes of Health. The government may have certain rights in this invention.

### FIELD OF THE INVENTION

The present invention relates to the field of anxiety and fear disorders, particularly to treating such disorders through inhibition of α-2,8-sialyltransferase activity.

### BACKGROUND OF THE INVENTION

The hippocampus and amygdala are involved in conditioned fear behavior. It is widely accepted that the amygdala is involved in fear memory (LeDoux, Annu. Rev. Neurosci. (2000) 23:155-184; Maren, Annu. Rev. Neurosci. (2001) 24:897-931). Fear associating with a single cue (a tone) paired with electrical shock requires the amygdala but not the hippocampus. However, fear associating the environment (the context) with shock requires both the amygdala and dorsal hippocampus (LeDoux, 2000, supra; Anagnostaras et al., Hippocampus (2001) 11:8-17; Maren, 2001, supra). The passive avoidance task generally requires multiple neurotransmitter systems throughout the brain including the amygdala and hippocampus (Ammassari-Teule et al., Brain Res. (1991) 551:104-109). There remains a need for effective therapies and agents for amelioration of anxiety and fear disorders.

### BRIEF SUMMARY OF THE INVENTION

The present invention provides compounds and methods for ameliorating a symptom of anxiety, fear, or depression behavior in a mammal in need thereof by decreasing or inhibiting α-2,8-sialyltransferase activity, particularly ST8Sia-II sialyltransferase activity, in the mammal. This decrease in activity may be attained in a number of ways including, but not limited to, administering a compound that directly or indirectly decreases or inhibits the enzymatic activity of the α-2,8-sialyltransferase. The compound can be, for example, an inactivating substrate analog of an α-2,8-sialyltransferase, for example, an analog of a donor saccharide (i.e, a CMP-sialic acid), and/or an analog of an acceptor saccharide (i.e., an N-linked glycan comprising at least one of a α-2,3-linked sialic acid, an α-2,6-linked sialic acid or an α-2,8-linked sialic acid). α-2,8-Sialyltransferase activity can also be decreased or inhibited through a decrease in transcription or translation of an α-2,8-sialyltransferase gene, a decrease in the RNA stability and/or half-life of a α-2,8-sialyltransferase transcript, and a decrease in stability and/or half-life of a α-2,8-sialyltransferase translated product. Agents that decrease or inhibit expression and/or the function of an α-2,8-sialyltransferase, or decrease or inhibit binding of ST8Sia-II-synthesized polysialic acid are thus of use in the methods of the invention. In certain embodiments, the methods preferentially decrease or inhibit the enzymatic activity of an ST8Sia-II enzyme in comparison to the inhibition of other α-2,8-sialyltransferases.

The invention further provides screening methods for identifying agents for use in reducing psychiatric disorders characterized by anxiety, fear and depression through in vitro and in vivo assays using a α-2,8-sialyltransferase gene, particularly an ST8Sia-II sialyltransferase gene, or functional segments thereof. This includes in vitro assays using a nucleic acid sequence encoding a α-2,8-sialyltransferase enzyme or amino acid sequences having α-2,8-sialyltransferase enzymatic activity, as well as in vivo assays using animal model systems, including mammalian model systems.

In one aspect, the invention provides in vitro methods for identifying a compound for use in reducing, inhibiting or preventing a psychiatric disorder characterized by anxiety, fear or depression by a) providing an assay mixture comprised of an α-2,8-sialyltransferase, a potential anxiety response modulator (i.e., inhibitor and/or enhancer), a cytidine 5′-monophosphate-N-acetylneuraminic acid (CMP-sialic acid) donor saccharide, an acceptor saccharide, and additional reagents required for α-2,8-sialyltransferase activity, b) incubating the assay mixture under conditions in which the α-2,8-sialyltransferase is active, and c) determining whether the amount of sialic acid transferred to the acceptor saccharide is increased or decreased in comparison to an assay mixture which lacks the potential anxiety response modulator. In such an assay, a potential anxiety response modulator which results in a decrease in sialic acid transfer to the acceptor saccharide is suitable for reducing, inhibiting or preventing a psychiatric disorder characterized by anxiety, fear or depression.

In one aspect, the invention provides methods for identifying compounds for decreasing, inhibiting or preventing psychiatric disorder characterized by anxiety, fear or depression in a mammal by a) providing a cell which comprises a polynucleotide that encodes an α-2,8-sialyltransferase, an acceptor saccharide for the α-2,8-sialyltransferase, and CMP-sialic acid, b) contacting the cell with a potential psychiatric disorder (i.e., anxiety, fear, or depression) modulator, c) incubating the cell under conditions in which the α-2,8-sialyltransferase is normally expressed; and d) determining whether the polysialic acid (PSA) level is increased or decreased compared to the PSA level in the absence of the potential psychiatric disorder modulator. In such an assay, a potential psychiatric disorder modulator that causes a decrease in the amount of PSA produced is useful for decreasing, inhibiting or preventing a psychiatric disorder characterized by anxiety, fear or depression in a mammal.

In one aspect, the invention provides methods for identifying lead compounds that decrease, inhibit or prevent a psychiatric disorder characterized by anxiety, fear or depression in a mammal, by contacting a library of potential anxiety response modulator compounds with an α-2,8-sialyltransferase and identifying potential anxiety response modulator compounds that bind to the α-2,8-sialyltransferase. A compound that binds to the α-2,8-sialyltransferase is a lead compound suitable for further testing as an anxiety response modulator. In one embodiment, the method comprises testing the lead compound by administering the compound to a test animal and determining whether the symptoms of the psychiatric disorder characterized by anxiety, fear or depression by the test animal to a stimulus is reduced or prevented by administration of the compound.

The invention also provides non-human transgenic mammals and inbred strains, including mice, for the study of anxiety and fear behavior. The transgenic mammals are characterized by the absence of the expression (i.e., by transcription or translation) of a functional α-2,8-sialyltransferase.

### DEFINITIONS

The following abbreviations are used herein:
Ara=arabinosyl;
Gal=galactosyl;
GalNAc=N-acetylgalactosaminyl;
Glc=glucosyl;
GlcNAc=N-acetylglucosaminyl;
NeuAc=sialyl (N-acetylneuraminyl).
PSA=polysialic acid
CMP=cytidine 5′-monophosphate

The term “sialic acid” refers to any member of a family of nine-carbon carboxylated sugars. The most common member of the sialic acid family is N-acetyl-neuraminic acid (2-keto-5-acetamindo-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al. (1990) J. Biol. Chem. 265: 21811-21819. Also included are 9-substituted sialic acids such as a 9-O-C1-C6 acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki (1992) Glycobiology 2: 25-40; Sialic Acids: Chemistry, Metabolism and Function, R. Schauer, Ed. (Springer-Verlag, New York (1992). The synthesis and use of sialic acid compounds in a sialylation procedure is disclosed in international application WO 92/16640, published Oct. 1, 1992.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right. All oligosaccharides described herein are described with the name or abbreviation for the non-reducing saccharide (e.g., Gal), followed by the anomeric configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2,3,2-3, 2→3, or (2,3). Each saccharide is a pyranose.

Much of the nomenclature and general laboratory procedures required in this application can be found in Sambrook, et al., Molecular Cloning: A Laboratory Manual (3rd Ed.), Vol. 1-3, Cold Spring Harbor Laboratory, Cold Spring Harbor, N.Y., 2000. The manual is hereinafter referred to as “Sambrook et al.” Procedures can also be found in Current Protocols in Molecular Biology, Eds. Ausubel, Brent, Kingston, Moore, Seidman, Smith, and Struhl, Updated April 2003, John Wiley & Sons, DOES Institute, Winston-Salem, N.C.

As used herein, “a psychiatric disorder characterized by anxiety, fear or depression” includes chronic or acute symptoms of anxiety, fear, depression, negative mood, pessimism, etc. Exemplified clinically defined psychiatric disorders treatable by the present invention include panic-agoraphobia (i.e., fear of open or public places) syndrome, panic syndromes, severe phobias, generalized anxiety disorder, social anxiety disorder, post-traumatic stress syndrome, obsessive-compulsive disorder and related disorders of impulse control. The methods also find use in treating major depression and severe, vital or “melancholic” depression. Psychiatric disorders, including depression and anxiety disorders, amenable to treatment by reducing, inhibiting or preventing α-2,8-sialyltransferase activity are reviewed in Chapter 19 of Goodman and Gilman's The Pharmacological Basis of Therapeutics, 10th Edition, Hardman, Limbird and Goodman-Gilman Eds., 2001, McGraw-Hill, the disclosure of which is hereby incorporated herein by reference.

“Sialyltransferases” refer to a family of glycosyltransferase enzymes that add sialic acid residues during oligosaccharide diversification (reviewed in Harduin-Lepers, et al., Glycobiology (1995) 5:741-758). Sialic acid addition occurs in the Golgi apparatus. Sialyl-transferases are classified under IUBMB Enzyme Nomenclature as E.C. 2.4.99.

As used herein, the term “α-2,8-sialyltransferase” refers to a sialyltransferase enzyme classified under IUBMB Enzyme Nomenclature as E.C. 2.4.99.8 and possessing α-2,8-sialyltransferase activity, also referred to as an alpha-N-acetylneuraminate alpha-2,8-sialyltransferase, and includes sialyltransferases “ST8Sia-I,” “ST8Sia-II” or “STX,” “ST8Sia-III,” “ST8Sia-IV” or “PST-1,” and “ST8Sia-V.”

As used herein, the terms “α-2,8-sialyltransferase activity” and “ST8Sia-II sialyltransferase activity” refers to the transfer or synthesis of a polysialic acid (PSA) moiety onto a glycoprotein or a glycolipid. Typically, a α-2,8-sialyltransferase catalyzes the synthesis of a α-2,8-linked sialic acid moiety from an activated cytidine 5′-monophosphate (CMP)-sialic acid to an acceptor saccharide residue on a glycoprotein (i.e., neural cell adhesion molecule or NCAM) or a glycolipid (i.e, ceramide). When initially transferring one or more α-2,8-linked sialic acid moieties onto a glycoprotein in an “initiase reaction,” the acceptor saccharide is typically an asparagine (N-) linked glycan comprising at least one of an α-2,3-linked sialic acid residue or an α-2,6-linked terminal sialic acid residue. When subsequently transferring one or more α-2,8-linked sialic acid moieties onto a glycoprotein in an “elongase reaction”, the acceptor saccharide is typically an asparagine (N-) linked glycan comprising at least one α-2,8-linked terminal sialic acid residue. “Initiase” and “elongase” reactions in polysialic acid synthesis catalyzed by α-2,8-sialyltransferases are summarized in Chapter 16 of Essentials of Glycobiology, Varki, Cummings, Esko, Freeze, Hart, and Marth Eds., 1999, Cold Spring Harbor Laboratory Press, Plainview, N.Y., the disclosure of which is hereby incorporated herein by reference.

As used herein, “polysialyltransferase activity” refers to the transfer of a α-2,8-linked sialic acid moiety to an acceptor saccharide comprising an asparagine (N-) linked glycan comprising at least one α-2,8-linked terminal sialic acid residue. Exemplified polysialyltransferases include ST8Sia-II and ST8Sia-IV.

The term “modulate” refers to the capacity to increase or decrease a response based on a known interaction, for instance, between a receptor and a ligand or an enzyme and a substrate. The term “modulator” refers to a compound, including a small molecule compound, including organic molecules and amino acid sequences, that can increase and/or decrease a known interaction, for instance between a receptor and a ligand or between an enzyme and a substrate. A modulator can modulate either directly or indirectly. A modulator can be an agonist, an antagonist, or a partial agonist/antagonist. A modulator can be a substrate analog.

As used herein, the term “substrate analog” refers to a compound that shares structural and/or functional similarity with an enzyme substrate, but unlike the enzyme substrate, the substrate analog inhibits the function of the enzyme upon binding. A substrate analog can have structural similarity to an enzyme substrate as measured on a 2-dimensional or 3-dimensional (electron densities, location of charged, uncharged and/or hydrophobic moieties) basis. A substrate analog can have functional similarity with an enzyme substrate inasmuch as the substrate analog binds to the enzyme. A substrate analog can be either a competitive or a non-competitive inhibitor.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

An “inhibitory nucleic acid” is any nucleic acid or modified nucleic acid used or designed for use in inhibitory nucleic acid therapy. “Inhibitory nucleic acid therapy” refers to the use of inhibitory nucleic acids to inhibit gene expression, for example, inhibition of DNA transcription, inhibition of RNA processing, transport or translation, or inhibition of protein synthesis. Inhibitory nucleic acid therapy includes the variety of approaches for treatment of disease using nucleic acids or modified nucleic acids as described herein. Various inhibitory nucleic acid therapies, including antisense nucleic acids, are discussed in detail below.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “heterologous sequence” or a “heterologous nucleic acid”, as used herein, is one that originates from a source foreign to the particular host cell, or, if from the same host cell, is modified from its original form.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, that includes nucleic acid elements that are capable of affecting expression of a structural gene in hosts that are compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “isolated” is meant to refer to material which is substantially or essentially free from components which normally accompany the enzyme or other material of interest as found in its native state. Thus, the enzymes, nucleic acids, or other materials of the invention, when isolated, do not include materials normally associated with their in situ environment. Typically, isolated proteins or nucleic acids are at least about 80% pure, usually at least about 90%, and preferably at least about 95% pure as measured by standard methods, such as by determining band intensity on a silver stained gel or other method for determining purity. Protein and nucleic acid purity or homogeneity can be indicated by a number of means well known in the art, such as polyacrylamide gel electrophoresis of a protein sample, followed by visualization upon staining. For certain purposes high resolution will be needed and HPLC or a similar means for purification utilized.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 70%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/) (see Henikoff & Henikoff, Proc. Natl. Acad. Sci. USA 89:10915 (1989)).

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

“Bind(s) substantially”, in the context of nucleic acids, refers to complementary hybridization between a probe nucleic acid and a target nucleic acid and embraces minor mismatches that can be accommodated by reducing the stringency of the hybridization media to achieve the desired detection of the target polynucleotide sequence.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium (as the target sequences are generally present in excess, at Tm, 50% of the probes are occupied at equilibrium).

The phrases “specifically binds” refers to a binding reaction which is determinative of the presence of the target molecule in the presence of a heterogeneous population of proteins, saccharides and other biologics. Thus, under designated assay conditions, the specified receptors or other compounds bind preferentially, for example, to a polysialic acid (PSA) or a ST8Sia-II polysialyltransferase or other molecule of interest and do not bind in a significant amount to other molecules present in the sample.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W.H. Freeman and Company.

The term “transgenic” refers to a cell that includes a specific genetic modification that was introduced into the cell, or an ancestor of the cell. Such modifications can include one or more point mutations, deletions, insertions, or combinations thereof. When referring to an animal, the term “transgenic” means that the animal includes cells that are transgenic, and descendants of such animals. An animal that is composed of both transgenic and non-transgenic cells is referred to herein as a “chimeric” animal.

### BRIEF DESCRIPTION OF THE DRAWINGS

FIG. 1 depicts mutagenesis of the ST8Sia-II gene. FIG. 1a is a schematic showing wild-type ST8Sia-II allele for ST8Sia-II, pflox targeting vector, mutant allele after homologous recombination in ES cells, and type I deleted (A) allele, represented with restriction enzyme sites. Polymorphism of BamHI site is found and denoted by an asterisk. Black boxes are exons and triangles indicate loxP sequences. E, EcoRI; H, HindIII; X, XhoI. FIG. 1b depicts genotypes determined by Southern blot analysis of genomic DNA from ES cells and mouse tail tissue digested by BamHI. FIG. 1c (top panels) depicts total RNA isolated from the brains of wild-type (WT), heterozygous (WT/Δ), and homozygous (Δ/Δ) mutant mice and used in RT-PCR with specific primer pairs for ST8Sia-II and ST8Sia-IV. RT-PCR analysis was also performed at 4 different ages (lower panels), postnatal day 0 (0), 1 month old (1M), 3 months old (3M), and 6 months old (6M).

FIG. 2 depicts Western blot analysis using anti-polysialic acid (PSA) antibody 5A5, and anti-NCAM antibody H28 of total protein extracts of different regions of the 8 week-old mouse brain from wild-type (wt/wt), heterozygous ST8Sia-II mutant (wt/Δ), and homozygous ST8Sia-II mutant (Δ/Δ) littermates. A portion of the sample was digested with endo-N before the analysis. OB, olfactory bulb; HY, hypothalamus; HP, hippocampus; CX, cerebral cortex; CB, cerebellum.

FIG. 3 depicts polysialic acid staining of the hippocampus and subventricular zone of wild-type and ST8Sia-II deficient mice. Cryosections of hippocampus from wild-type (WT, a and b) and ST8Sia-II deficient (Δ/Δ, c and d) mice were stained with anti-PSA antibody, 12F8. Nissl staining is indicated in green and PSA in red. In WT mice, granule cells in the dentate gyrus are PSA-positive (arrowheads in b). In contrast to WT mice, note that precursor cells in the dentate gyrus are not labeled by 12F8 in Δ/Δ mice (d). PSA (red) was detected in subventricular zone from WT (e-g) and Δ/Δ(h-j) and nuclei (blue) were shown by Hoechst staining (e and h). Panels f, g, i, and j are magnifications of e and h as indicated. Migrating cells are PSA-positive in both genotypes (arrowheads in f and i). However, Δ/Δ has a few cells expressing PSA near anterior part of subventricular zone (arrowheads in j) compared to PSA-positive cells in WT(g). To label mitotic cells in embryonic and adult brains, BrdU was injected into a pregnant mouse at embryonic day 16 (k and n) and into adult mice at 2-3 months post-natal age (l, m, o, p), respectively. BrdU-labeled cells were visualized by anti-BrdU antibody (green) and nuclei were stained with Hoechst dye (blue). CC, corpus callosum; DG, dentate gyrus; G, granule cell layer; H, hilus; LV, lateral ventricle; S, striatum.

FIG. 4 depicts hippocampal mossy fiber topology. Timm's staining was performed (a-h) on brain tissue from denoted genotypes at various developmental stages, 2 weeks old (2W), 1 month old (1M), 3 months old (3M), and 6 months old (6M). From early in development, the infrapyramidal mossy fiber projection in ST8Sia-II-deficient mice is larger than in wild-type mice and extends into the CA3a region (arrows). Hippocampal sections from wild-type and ST8Sia-II deficient mice (3 months old) were also stained with anti-calbindin (CaBP, i and j) and anti-NCAM (k and 1) antibodies.

FIG. 5 depicts synapse formation in the stratum oriens of the hippocampal CA3 region. FIG. 5 a) PSA is expressed in suprapyramidal and infrapyramidal (arrowheads) mossy fibers of wild-type and ST8Sia-II deficient mice. The pyramidal cell layer also expresses polysialic acid (arrows). FIG. 5b) Synapse formation was compared by immunohistochemical staining with anti-synapsin I antibodies among wild-type (a-c) and ST8Sia-II deficient (d-f) mice. b and e are views of CA3c using higher magnification of boxed regions in a and d, respectively. Ectopic synapses (white arrows) are found in the ST8Sia-II-deficient mice. The CA3c region of wild-type (c) and ST8Sia-II mutant (f) mice also show difference in synapse formation.

FIG. 6 depicts short- and long-term potentiation in the hippocampal CA1 and CA3 regions. FIG. 6a) Theta-burst stimulation (TBS) of Schaffer collaterals (marked by arrow) evoked a strong increase in the slopes of fEPSPs recorded in the CA1 region of wild-type mice and in ST8Sia-II-deficient mice. Traces on the top show averaged fEPSPs recorded before and 50-60 minutes after induction of LTP in wild-type and ST8Sia-II deficient mice. Scale bars, 10 ms and 500 μV. FIG. 6b) Single train of high-frequency stimulation of mossy fibers (1×HFS, marked by arrow) evoked a similar increase in amplitude of fEPSPs in the CA3 region of wild-type and ST8Sia-II deficient mice. Traces on the top show averaged fEPSPs recorded before and 50-60 min after induction of LTP. Scale bars, 10 ms and 50 μV. In a-b, data shown are means±SEM. n indicates the number of slices and N indicates the number of mice.

FIG. 7 depicts metabolic and behavioral analyses. FIG. 7a) ST8Sia-II deficient mice displayed enhanced exploratory behavior in the open field test. The total distance traveled in a 30 min analysis was significantly increased in the ST8Sia-II deficient mice (t_{[48]}=2.65, p<0.01). The distance traveled in the center of the arena was also greater in the ST8Sia-II deficient mice (t_{[48]}=2.9, p<0.005). There was a trend for the ratio of the distance traveled in the center by the total distance traveled to be increased in the ST8Sia-II Δ/Δ mice p<0.08). FIG. 7b) In the passive avoidance test, the latency to enter the chamber was decreased in ST8Sia-II deficiency (t_{[47]}=2.79, p<0.005), suggesting impaired memory for the earlier noxious stimulus. ST8Sia-II deficient mice were also impaired in both the amygdale dependent cued version of the conditioned fear task (t_{[48]}=2.56, p<0.01—the three tone only trials were collapsed for analysis) and the amygdala and hippocampal dependent contextual fear conditioning (t_{[48]}=2.23, p<0.05). FIG. 7c) Acoustic startle analysis indicated normal responses. FIG. 7d) In the water maze test, a repeated measures ANOVA analysis showed that both genotypes improved similarly across blocks of trials in the hidden platform (acquisition) trials and the t-test showed that the groups did not differ significantly in their probe trial performance. FIG. 7e) Automated analyses with metabolic cages and repeated ANOVA measures of the data revealed normal motor activity, food and water consumption, and caloric values (wt/wt, while circles; Δ/Δ black circles). There was no evidence of basal hyperactivity.

### DETAILED DESCRIPTION

General

It has been discovered that polysialic acid (PSA) formation by an α-2,8-sialyltransferase, and in collaboration with neural cell adhesion molecule (NCAM), is an important determinant in the formation of neural circuits operating in the acquisition of fear behavior. A deficiency in expression of an ST8Sia-II sialyltransferase alters fear responses normally processed by both the hippocampus and amygdala. The hippocampus and amygdala are involved in conditioned fear behavior. The unique spectrum of effects in α-2,8-sialyltransferase deficiency is associated with an increase in exploratory behavior and reduced responses to fear conditioning in mice. The present invention provides methods and agents for treating symptoms of anxiety, fear, and depression and other psychiatric disorders that pertain to anxiety and fear behavior.

### Detailed Embodiments

The present invention provides methods and compounds that are suitable for ameliorating (i.e., decreasing, inhibiting or preventing) psychiatric disorders characterized by anxiety, fear or depression. Such methods and compounds can be used, prophylactically, chronically or acutely, to reduce, inhibit or prevent psychiatric disorders characterized by anxiety, fear or depression. Also provided are screening methods for identifying compounds that are useful for decreasing, inhibiting or preventing a psychiatric disorder characterized by anxiety, fear or depression. Such compounds are suitable for use directly, or for use as lead compounds to identify further compounds that are useful for decreasing, inhibiting or preventing psychiatric disorders characterized by anxiety, fear or depression. Transgenic animals that lack a functional gene for an α-2,8-sialyltransferase are also provided by the invention. In a preferred embodiment of the invention, the methods preferentially decrease, inhibit or prevent the polysialyltransferase activity of an ST8Sia-II α-2,8-sialyltransferase in comparison to other α-2,8-sialyltransferases.

The invention is based in part on the surprising discovery that mice with a non-functional ST8Sia-II gene exhibit reduced fear conditioning and anxiety responses and increased exploratory behavior. Accordingly, the methods of the invention include reducing, inhibiting or preventing a psychiatric disorder characterized by symptoms of anxiety, fear or depression by administering to a mammal in need thereof a therapeutically effective amount of one or more compounds that decrease or inhibit α-2,8-sialyltransferase activity. The compounds can reduce or prevent the synthesis of, or enhance the degradation of, polysialic acid (PSA) on substrate glycoproteins, including NCAM and/or glycolipids, including ceramide. The compounds can reduce or inhibit the transcription or translation of an α-2,8-sialyltransferase gene, thereby reducing or inhibiting the production of a functional α-2,8-sialyltransferase enzyme. The compounds can directly or indirectly decrease or inhibit the enzymatic activity of a α-2,8-sialyltransferase. In certain embodiments, the compounds are substrate analogs of an α2,8-sialyltransferase, for example, analogs of a donor saccharide or analogs of an acceptor saccharide. The mammal can be a non-human mammal, including canine, feline, porcine, bovine, ovine, murine, rodentia and lagomorpha. The mammal is typically a human. Hippocampal synaptic plasticity, as measured by long-term potentiation (LTP), short-term potentiation (STP), post-tetanic potentiation (PTP) and/or excitatory post-synaptic potentials (ESPS), is not significantly altered by the loss or blocking of a functional α-2,8-sialyltransferase, particularly a ST8Sia-II α-2,8-sialyltransferase.

The methods and compositions of the invention alter the amount of polysialic acid that is present and available for binding on various cell surface proteins, for example NCAM, and for influencing cell-cell communication involving integrins, cadherins, and members of the immunoglobulin super-family (see, Rutishauser, J. Cell Biochem (1998) 70:304-312; Fujimoto, et al., J. Biol Chem (2001) 276:31745-31751) The amount of PSA available on various cells types, for example, on neuronal progenitor cells and on hippocampal granule cells, can also be altered by the loss or blocking of a functional α-2,8-sialyltransferase. α-2,8-sialyltransferases direct the synthesis of α2-8 linked sialylated structures as any of several glycoconjugates on glycoproteins or glycolipids. When attached to a glycoprotein, polysialic acid is generally linked to the protein through an asparagine-(N-) linked glycan to an asparagine residue. The polysialic acid can be composed of 100 or more sialic acid residues. N-glycans with terminal sialic acid moieties at least one of an α2-3 linkage and an α2-6 linkage serve as substrates (i.e., acceptors) for the attachment of an initial α2-8 linked sialic acid residue, and an α-2,8-sialyltransferase can further catalyze the elongation of α2-8 linked sialic acid residue chains. For example, an α-2,8-sialyltransferase can transfer multiple α2-8 linked sialic acid residues to an acceptor N-glycan containing a NeuAc α2-3 (or α2-6) Gal β1-4GlcNAc β1-R structure. α-2,8-sialyltransferase activity is reviewed, for example, in Angata and Fukada, Biochimie (2003) 85:195-206; and in Chapter 16 of Essentials of Glycobiology, supra, the disclosures of each of which are hereby incorporated herein by reference.

In some embodiments, the methods of the invention cause a change in the activity of an α-2,8-sialyltransferase. This enzyme catalyzes the synthesis of a polysialic acid moiety from the activated donor sugar cytidine 5′-monophosphate (CMP)-sialic acid residues to an acceptor saccharide that can include at least one of an α-2,3-linked, an α-2,6-linked or an α-2,8-linked terminal sialic acid moiety. Accordingly, the invention includes blocking agents that decrease or inhibit the activity of a α-2,8-sialyltransferase, particularly an ST8Sia-II sialyltransferase. The blocking agents of the invention can act directly on the enzyme, or act as a substrate for the enzyme, for instance as an inactivating substrate analog of the enzyme. The blocking agents can also decrease or inhibit the expression of a gene that encodes the enzyme, at either or both the transcriptional and translational levels.

Methods are also disclosed for preparing the psychiatric disorder modulating agents as well as various screening assays to identify suitable candidates. Therapeutic and other uses for these compounds are also provided.

I. Methods for Modulating α-2,8-Sialyltransferase-Mediated Anxiety, Fear or Depression Responses

The present invention provides methods for modulating the synthesis and/or transfer of polysialic acid moieties to glycoproteins on cells that are involved in anxiety, fear and/or depression behaviors. Certain anxiety, fear and/or depression behaviors are mediated in part by polysialic acid expression on cells in regional areas of the brain associated with neural stem cell genesis and/or neurogenesis, for example, hippocampal granule cells, neuronal progenitor cells in the dentate gyrus and neural precursor cells along the lateral ventricle in the subventricular zone of the brain. By decreasing, inhibiting or preventing the synthesis of polysialic acid moieties to glycoproteins on cells in regional areas of the brain where neural stem cell genesis and/or neurogenesis occurs, undesirable anxiety, fear, and/or depression behaviors can be reduced, inhibited or prevented.

A. Inhibitors of α-2,8-Sialyltransferases

In one embodiment, the methods involve reducing, inhibiting or preventing a psychiatric disorder characterized by anxiety, fear or depression by inhibiting the enzymatic activity of an α-2,8-sialyltransferase, particularly a ST8Sia-II α-2,8-sialyltransferase. Preferably, the inhibitor preferentially inhibits the enzymatic activity of an α-2,8-sialyltransferase in comparison to inhibiting the enzymatic activity of other sialyltransferases, for example, α-2,3-sialyltransferases or α-2,6-sialyltransferases. In one embodiment, the inhibitor preferentially inhibits the enzymatic activity of an ST8Sia-II (STX) enzyme in comparison to the enzymatic activity of other α-2,8-sialyltransferases, for example, an ST8Sia-IV (PST-1) sialyltransferase.

Having identified the target enzyme to be inhibited (e.g. a α-2,8-sialyltransferase or a ST8Sia-II sialyltransferase), many approaches can be used to block its activity. Examples of agents capable of inhibiting enzyme activity include substrate analogs, suicide substrates, alkylating agents, and inhibitory nucleic acids (reviewed in Ferscht, Structure and Mechanism in Protein Science: A Guide to Enzyme Catalysis and Protein Folding, 3rd Edition, 1999, W.H. Freeman & Co.). The methods of decreasing, inhibiting or preventing α-2,8-sialyltransferase activity can involve administering to a subject, including a mammal such as a human, a compound that is an analog of a substrate for an α-2,8-sialyltransferase, including a donor saccharide and/or an acceptor saccharide. In certain embodiments, the methods involve decreasing, inhibiting or preventing ST8Sia-II sialyltransferase activity by administering to a subject an analog of a preferential substrate for an ST8Sia-II sialyltransferase in comparison to other α-2,8-sialyltransferases.

In some embodiments, the inhibitor is a sugar nucleotide or an analog of a donor substrate, e.g., an analog of sialic acid or CMP-sialic acid. As discussed above, the donor substrate of sialyl transferases are sugar nucleotides, usually monophosphonucleosides. For example, cytidine monophosphosugars are donor substrates for the formation of glycosides of sialic acid by α-2,8-sialyltransferases.

Using this knowledge, one of skill in the art can readily synthesize a number of sugar nucleotides which can then be tested to identify those capable of maximum inhibition of a specific enzyme. The term “sugar nucleotide” as used herein refers both to sugar nucleotides discussed above and to various analogs thereof that might be synthesized or isolated from natural sources. The number of variations on this structure is limitless. For instance, both the ester linkage between the sugar and phosphate and the anhydride linkage of the pyrophosphate are potential targets of enzymatic cleavage. Replacement of the O—P or C—O linkage with a more stable C—P bond provides nucleotide monophosphate or diphosphate sugar analogs that are more resistant to enzymatic degradation. Such compounds have the potential to selectively inhibit glycoprotein or glycolipid synthesis by acting as substrate analogs of a particular α-2,8-sialyltransferase. See, e.g., Vaghefi et al., J. Med. Chem. 30:1383-1391 (1987), and Vaghefi et al., J. Med. Chem. 30:1391-1399 (1987).

Another approach is to replace the monophosphate or diphosphate bridge between the sugar residue and the nucleoside moiety. For instance, the diphosphate bridge can be replaced with an isosteric —OCONHSO₂O— residue. See, Samarasa, et al., J. Med. Chem. 28:40-46 (1985).

Analogs of sugar nucleotides capable of inhibiting glycosylation have been used as antibiotics and antiviral agents. Examples of such compounds include 2-deoxy-D-glucose, which is transformed to either UDP-2dGlc or GDP-2dGlc and in that form inhibits glycosylation of glycoproteins in the viral envelope. DeClercq, Biochem. J. 205:1 (1982), which is hereby incorporated herein by reference. Antibiotics such as tunicamycin and streptovirudin are also effective because of their ability to inhibit glycosylation. For instance, tunicamycin is an analog of UDP-GlcNAc, the donor substrate for N-acetylglucos-aminyltransferases. The replacement of diphosphate bridge with a carbon chain allows tunicamycin to cross the cell membrane but still readily bind the active site of the enzyme. Examples of cytidine analog drugs are disclosed in Chapter 52 of Goodman and Gilman, supra, the disclosure of which is hereby incorporated herein by reference. The structure of these and related compounds provide one of skill in the art with direction in designing and synthesizing compounds with similar inhibitory effects in accordance with the present invention as described herein.

Nucleotides are the byproduct of the reaction by which sialyl residues are transferred to acceptor substrates. Nucleotides have been found to competitively inhibit sialyltransferases. Thus, various nucleotides and their analogs have potential as inhibitors of these enzymes. For example, cytidine monophosphate (CMP), cytidine diphosphate (CDP) or cytidine triphosphate (CTP) can be used to inhibit α-2,8-sialyltransferase activity. Further, compounds that affect intracellular pools of CTP (i.e., 3-deazauridine, acivicin, and 1-beta-D-arabinofuranosylcytosine) have been shown to curtail cell surface membrane resialylation (see, Hindenburg, et al., Cancer Res. (1985) 45:3048-52, hereby incorporated herein by reference).

In addition to the donor substrate analogs, analogs of acceptor substrates may also be used as inhibitors. Again, the skilled artisan will recognize a variety of possible structures that can be used. Because of the acceptor saccharide substrate specificity of at least one of an α-2,3-linked sialic acid or an α-2,6-linked sialic acid for initiation reactions or an α-2,8-linked sialic acid for elongation reactions, specific inhibition of an α-2,8-sialyltransferase initiation or elongation reaction can be achieved. Ideally, the inhibitory compounds should be capable of acting as specific acceptor substrates for a given α-2,8-sialyltransferase, even in the presence of other enzymes. In addition, the compound should be an efficient acceptor substrate. Thus, the Kᵢ of the inhibitor should be at least about 10⁻⁵ M, more preferably at least about 10⁻⁷ M. Suitable analogs for inhibition of an α-2,8-sialyltransferase elongation reaction include derivatives of α2,3-linked sialic acid and α2,6-linked sialic acid. Other suitable analogs of acceptor substrates include compounds that are converted or metabolized into sialic acid derivatives. The sialic acid acceptor saccharide derivatives bind to an α-2,8-sialyltransferase, but do not function as an acceptor, allowing for elongation of a α-2,8-linked sialic acid chain. For example, a small molecule converted to an unnatural sialic acid derivative (i.e., N-butanoylmannosiamine) has been shown to inhibit PSA expression on cultured neurons by effectively acting as a chain terminator (see, Mahal, et al., Science (2001) 294:380-81).

α-2,8-Sialyltransferases can also be inhibited by contacting acceptor substrates for the sialyltransferase with a competing sialyltransferase or glycosidase that converts the acceptor oligosaccharide into a different structure that does not function as an acceptor for the sialyltransferase of interest, for example an ST8Sia-II sialyltransferase.

Naturally occurring molecules which show inhibitory effects can also be isolated for use in the present invention. The biosynthesis of glycoproteins or glycolipids is a complex metabolic pathway that depends on many factors for regulation. Naturally occurring inhibitory compounds can be purified and used to further inhibit activity.

The preferred glycosyltransferase inhibitors of the present invention have the ability to cross the cell membrane and enter the Golgi apparatus. Thus, the blocking agents are preferably sufficiently hydrophobic to allow diffusion through the membrane. Generally, they have no other adverse effects on cellular metabolism, so that other glycosylation reactions proceed while the specific reaction is inhibited. The blocking agents are preferably relatively small molecules, thereby avoiding immunogenicity and allowing passage through the cell membrane. Ideally, they have a molecular weight of between about 100-2000 daltons, but may have molecular weights up to 5000 or more, depending upon the desired application. In most preferred embodiments, the inhibitors have molecular weights of between about 200-600 daltons.

The inhibitors of the present invention preferably have strong affinity for the target enzyme, so that at least about 60-70% inhibition of sialyltransferase activity is achieved, more preferably about 75%-85% and most preferably 90%-95% or more. In some embodiments, the inhibitors will completely inhibit sialyltransferase activity. The affinity of the enzyme for the inhibitor is preferably sufficiently strong that the dissociation constant, or Kᵢ, of the enzyme-inhibitor complex is less than about 10⁻⁵ M, typically between about 10⁻⁶ and 10⁻⁸ M.

Enzyme inhibition generally involves the interaction of a substance with an enzyme so as to decrease the rate of the reaction catalyzed by that enzyme. Inhibitors can be classified according a number of criteria. For example, they may be reversible or irreversible. An irreversible inhibitor dissociates very slowly, if at all, from its target enzyme because it becomes very tightly bound to the enzyme, either covalently or noncovalently. Reversible inhibition, in contrast, involves an enzyme-inhibitor complex which may dissociate.

Inhibitors can also be classified according to whether they are competitive, noncompetitive or uncompetitive inhibitors. In competitive inhibition for kinetically simple systems involving a single substrate, the enzyme can bind either the substrate or the inhibitor, but not both. Typically, competitive inhibitors resemble the substrate or the product(s) and bind the active site of the enzyme, thus blocking the substrate from binding the active site. A competitive inhibitor diminishes the rate of catalysis by effectively reducing the affinity of the substrate for the enzyme. Typically, an enzyme may be competitively inhibited by its own product because of equilibrium considerations. Since the enzyme is a catalyst, it is in principle capable of accelerating a reaction in the forward or reverse direction.

Noncompetitive inhibitors allow the enzyme to bind the substrate at the same time it binds the inhibitor. A noncompetitive inhibitor acts by decreasing the turnover number of an enzyme rather than diminishing the proportion of free enzyme. Another possible category of inhibition is mixed or uncompetitive inhibition, in which the inhibitor affects the binding site and also alters the turnover number of the enzyme. Enzyme inhibition of kinetically complex systems involving more than one substrate, as can be the case for α-2,8-sialyltransferase, are described in Segel, Enzyme Kinetics, (Wiley, N.Y. 1975).

α-2,8-Sialyltransferase activity and its inhibition or enhancement is typically assayed according to standard methods for determining enzyme activity. For a general discussion of enzyme assays, see, Rossomando, “Measurement of Enzyme Activity” in Guide to Protein Purification, Vol. 182, Methods in Enzymology (Deutscher ed., 1990). An assay for α-2,8-sialyltransferase activity typically contains a buffered solution adjusted to physiological pH, a source of divalent cations, a donor substrate (for example, a labeled CMP-sialic acid moiety), an acceptor substrate (e.g., a glycan including a terminal α-2,3-linked sialic acid and/or a terminal α-2,6-linked sialic acid), an α-2,8-sialyltransferase, and the sample or fraction of a sample whose inhibitory activity is to be tested. After a predetermined time at 23° C. or 37° C., the reaction is stopped and the sialylated product is isolated and measured according to standard methods (e.g., in a scintillation counter). When testing for the ability of a test compound to decrease or inhibit α-2,8-sialyltransferase activity, the sialyltransferase activity of an α-2,8-sialyltransferase exposed to the test compound is compared to the sialyltransferase activity of an α-2,8-sialyltransferase in a control unexposed to the test compound.

B. Inhibition of α-2,8-Sialyltransferase Gene Expression

Decreasing or inhibiting α-2,8-sialyltransferase gene expression can be achieved through the use of inhibitory nucleic acids. Inhibitory nucleic acids can be single-stranded nucleic acids that can specifically bind to a complementary nucleic acid sequence. By binding to the appropriate target sequence, an RNA-RNA, a DNA-DNA, or an RNA-DNA duplex or triplex is formed. Such inhibitory nucleic acids can be in either the “sense” or “antisense” orientation.

In one embodiment, the inhibitory nucleic acid can specifically bind to a target nucleic acid that encodes an α-2,8-sialyltransferase. Administration of such inhibitory nucleic acids can decrease or inhibit psychiatric disorders characterized by anxiety, fear or depression behaviors by reducing or eliminating the biosynthesis of polysialic acid on glycoproteins and/or glycolipids. Nucleotide sequences encoding an α-2,8-sialyltransferase are known for several species, including human. Human nucleic acid sequences encoding an ST8Sia-II (STX) sialyltransferase have been published as GenBank accession numbers NM_{—}006011, BC069584, and U82762. Nucleotide sequences from non-human species encoding an ST8Sia-II sialyltransferase have been published as GenBank accession numbers XM_{—}545840 (Canis familiaris), NM_{—}009181 (Mus musculus), AJ715538 (Takifugu rubripes-pufferfish), AJ715537 (Tetraodon nigroviridis-pufferfish), and AJ699419 (Gallus gallus-chicken). Nucleotide sequences encoding other α-2,8-sialyltransferases have been published as GenBank accession numbers NM_{—}005668, NM_{—}015879, NM_{—}175052, NM_{—}003034 and AF003092. From these nucleotide sequences, one can derive a suitable inhibitory nucleic acid.

By binding to the target nucleic acid, the inhibitory nucleic acid can inhibit the function of the target nucleic acid. This could, for example, be a result of blocking DNA transcription, processing or poly(A) addition to mRNA, DNA replication, translation, or promoting inhibitory mechanisms of the cells, such as promoting RNA degradation. Inhibitory nucleic acid methods therefore encompass a number of different approaches to altering expression of specific genes that operate by different mechanisms. These different types of inhibitory nucleic acid technologies are described in Helene and Toulme, (1990) Biochim. Bioplys. Acta., 1049:99-125.

Inhibitory nucleic acid therapy approaches can be classified into those that target DNA sequences, those that target RNA sequences (including pre-mRNA and mRNA), those that target proteins (sense strand approaches), and those that cause cleavage or chemical modification of the target nucleic acids.

Approaches targeting DNA fall into several categories. Nucleic acids can be designed to bind to the major groove of the duplex DNA to form a triple helical or “triplex” structure. Alternatively, inhibitory nucleic acids are designed to bind to regions of single stranded DNA resulting from the opening of the duplex DNA during replication or transcription. See Helene and Toulme, supra.

More commonly, inhibitory nucleic acids are designed to bind to mRNA or mRNA precursors. Inhibitory nucleic acids are used to prevent maturation of pre-mRNA. Inhibitory nucleic acids may be designed to interfere with RNA processing, splicing or translation. For example, an inhibitory sense or antisense nucleic acid complementary to regions of a target mRNA inhibits protein expression. See, e.g., Wickstrom E. L. et al. (1988) Proc. Nat'l. Acad. Sci. USA 85:1028-1032 and Harel-Bellan et al. (1988) Exp. Med., 168:2309-2318. Of particular interest are inhibitory nucleic acids introduced into the cell can also encompass segment of the “sense” strand of the gene or mRNA, e.g., short interfering or small inhibitor RNA sequences. Inhibitory nucleic acids targeting mRNA have been shown to work by several different mechanisms in order to inhibit translation of the encoded protein(s). Techniques for the regulation of expression by effectors that bind to RNA are reviewed, for example, in Helene and Toulme, supra, and in Grundy and Henkin, Curr Opin Microbiol (2004) 7:126-131, the disclosures of each of which are hereby incorporated herein by reference.

The inhibitory nucleic acids also can be used to induce chemical inactivation or cleavage of the target genes or mRNA. Chemical inactivation can occur by the induction of crosslinks between the inhibitory nucleic acid and the target nucleic acid within the cell. Alternatively, cleavage can be induced by the use of ribozymes or catalytic RNA. In this approach, the inhibitory nucleic acids would comprise either naturally occurring RNA (ribozymes) or synthetic nucleic acids with catalytic activity. In other embodiments, expression of α-2,8-sialyltransferase genes is inhibited by administration of an agent that blocks the ability of a transactivating factor to induce gene expression. The targeting of inhibitory nucleic acids to specific cells of interest by conjugation with targeting moieties binding receptors on the surface of these cells can be used for all of the above forms of inhibitory nucleic acid therapy.

C. Compounds that Block Polysialic Acid Binding

The invention also provides methods of decreasing or inhibiting a psychiatric disorder characterized by anxiety, fear or depression in which a compound is administered that blocks the interaction between a polysialic acid moiety on a glycoprotein or a glycolipid and a ligand for the sialic acid moiety or for the glycoprotein or glycolipid on which the oligosaccharide is bound. It is of particular interest to block polysialic acid interactions in regions of the brain associated with neural stem cell genesis and/or neurogenesis, for example, the subventricular zone and the dentate gyrus, particularly at the border between the hilus and the granule cell layer. Cell types of interest for targeting of the blocking compounds include hippocampal granule cells, neuronal progenitor cells in the dentate gyrus (which migrate into the granule cell layer), and neural precursor cells along the lateral ventricle in the subventricular zone of the brain. For example, one can administer an antibody or lectin that specifically binds to the polysialic acid moiety or an antibody that specifically binds to the glycoprotein or glycolipid on which the polysialic acid is bound, directing the lectin or antibody to the desired regions of the brain and the particular cell types of interest. Receptors that are not associated with a target cell (e.g., that are present as free polysaccharides or glycoproteins) can be used to block the interaction between target cell-associated PSA and cellular receptors of the target cell. Other compounds, such as small molecules identified using the methods described herein, can also be administered.

Monoclonal antibodies against polysialic acid and monoclonal antibodies that specifically recognize α-2,8-linked polysialic acid chains have been developed (Nadasdy, et al., Hum Pathol. (1993) 24:413-9; Letaief, et al., Biol Cell (1993) 77:269-76). Sialic specific lectins include lobster lectin (LAgl) (VanderWall, et al., Dev Comp Immunol (1981) 5:679-83), wheat germ agglutinin (WGA) (Griffith and Wiley, Anat Enb yol (Berl.) (1991) 183:205-12), concavalin A (Con A), Sambucus nigra agglutinin and Maackia amurensis agglutinin (Letaief, et al., supra). For delivery, markers for neural precursor cells, for instance, nestin, vimentin, and BM88, and antibodies against such markers are known by those of skill in the art (see, for example, Koutmani, et al., Eur J Neurosci (2004) 20:2509; and Ko, et al., Biomaterials (2005) 26:687-96). Neural stem cells themselves can also serve as delivery vehicles of blocking agents to regions of the brain associated with neural stem cell genesis and/or neurogenesis (Kabos, et al., Expert Opin Biol Ther (2003) 3:759-70).

In one embodiment, the methods involve use of a small organic compound, a peptide or an antibody to decrease, inhibit, or prevent the homophilic binding of polysialylated NCAM molecules in regions of the brain associated with neural stem cell genesis and/or neurogenesis. Peptide ligands of NCAM have been developed and can serve as lead compounds that block the homophilic binding of polysialated NCAM without eliciting intracellular signaling (see, for example, Hartz, et al., Pharmacol Biochem Behav (2003) 75:861-7; Ronn, et al., Nat Biotechnol (1999) 17:1000-5; Ronn, et al., Eur J Neurosci (2002) 16:1720-30; and Kiryushko, et al., J Biol Chem (2003) 278:12325-34).

II Screening Methods

One can identify lead compounds that are suitable for further testing to identify those that are therapeutically effective modulating agents by screening a variety of compounds and mixtures of compounds for their ability to decrease, inhibit or enhance α-2,8-sialyltransferase activity, or which bind to a polysialic acid moiety and prevent the ligand from binding to its receptor. The testing can be performed using either a minimal core polysialic acid moiety, or a modified polysialic acid moiety to which additional saccharide residues (i.e., sialic acid residues) can be added. For example, one can test the ability of a lead compound to bind to a core 2 polysialic acid moiety having a terminal α-2,3-linked sialic acid and/or a terminal α-2,6-linked sialic acid residue.

The use of screening assays to discover naturally occurring compounds with desired activities is well known and has been widely used for many years. For instance, many compounds with antibiotic activity were originally identified using this approach. Examples of such compounds include monolactams and aminoglycoside antibiotics. Compounds which inhibit various enzyme activities have also been found by this technique, for example, mevinolin, lovastatin, and mevacor, which are inhibitors of hydroxymethylglutamyl Coenzyme A reductase, an enzyme involved in cholesterol synthesis. Antibiotics that inhibit glycosyltransferase activities, such as tunicamycin and streptovirudin have also been identified in this manner.

Thus, another important aspect of the present invention is directed to methods for screening samples for α-2,8-sialyltransferase modulating activity, particularly for α-2,8-sialyltransferase reducing or inhibiting activity. A “sample” as used herein may be any mixture of compounds suitable for testing in a α-2,8-sialyltransferase assay. A typical sample comprises a mixture of synthetically produced compounds or alternatively a naturally occurring mixture, such as a cell culture broth. Suitable cells include any cultured cells such as mammalian, insect, microbial or plant cells. Microbial cell cultures are composed of any microscopic organism such as bacteria, protozoa, yeast, fungi and the like.

In the typical screening assay, a sample, such as a fungal broth, is added to a standard α-2,8-sialyltransferase assay. If inhibition or enhancement of activity as compared to control assays is found, the mixture is usually fractionated to identify components of the sample providing the inhibiting or enhancing activity. The sample is fractionated using standard methods such as ion exchange chromatography, affinity chromatography, electrophoresis, ultrafiltration, HPLC and the like. See, e.g., Protein Purification, Principles and Practice, (Springer-Verlag, 1982). Each isolated fraction is then tested for inhibiting or enhancing activity. If desired, the fractions are then further subfractionated and tested. This subfractionation and testing procedure can be repeated as many times as desired.

By combining various standard purification methods, a substantially pure compound suitable for in vivo therapeutic testing can be obtained. A substantially pure modulating agent as defined herein is an activity inhibiting or enhancing compound which migrates largely as a single band under standard electrophoretic conditions or largely as a single peak when monitored on a chromatographic column. More specifically, compositions of substantially pure modulating agents will comprise less than ten percent miscellaneous compounds.

In preferred embodiments, the assays are designed to screen large chemical libraries by automating the assay steps and providing compounds from any convenient source to assays, which are typically run in parallel (e.g., in microtiter formats on microtiter plates in robotic assays).

As noted, the invention provides in vitro assays for α-2,8-sialyltransferase activity in a high throughput format. For each of the assay formats described, “no modulator” control reactions which do not include a modulator provide a background level of α-2,8-sialyltransferase activity. In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100 about 1500 different compounds. It is possible to assay many different plates per day; assay screens for up to about 6,000-20,000, and even up to about 100,000-1,000,000 different compounds is possible using the integrated systems of the invention. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

In some assays it will be desirable to have positive controls to ensure that the components of the assays are working properly. For example, a known modulator of α-2,8-sialyltransferase activity can be incubated with one sample of the assay, and the resulting increase or decrease in signal determined according to the methods herein.

Essentially any chemical compound can be tested as a potential modulator of α-2,8-sialyltransferase activity for use in the methods of the invention. Most preferred are generally compounds that can be dissolved in aqueous or organic (especially DMSO-based) solutions are used. It will be appreciated that there are many suppliers of chemical compounds, including Sigma (St. Louis, Mo.), Aldrich (St. Louis, MO)), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

In one preferred embodiment, modulators of α-2,8-sialyltransferase activity or of binding to polysialic acid moieties are identified by screening a combinatorial library containing a large number of potential therapeutic compounds (potential modulator compounds). Such “combinatorial chemical or peptide libraries” can be screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furla, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/100091), benzodiazepines (U.S. Pat. No. 5,288,514), diversomers such as hydantoins, benzodiazepines and dipeptides (Hobbs et al., Proc. Nat. Acad. Sci. USA 90:6909-69L 3 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

IIL Therapeutic and Diagnostic Uses of the Invention

The compositions and methods of the present invention can be used therapeutically to selectively reduce, inhibit or prevent α-2,8-sialyltransferase activity or binding of an ST8Si-1H-synthesized polysialic acid moiety, particularly the activity of an ST8Sia-II sialyltransferase, that is associated with a variety of conditions such as psychiatric disorders characterized by anxiety, fear or depression. Fear, mood and anxiety disorders suitable for treatment by reducing, inhibiting or preventing α-2,8-sialyltransferase activity include panic-agoraphobia (i.e., fear of open or public places) syndrome, severe phobias, generalized anxiety disorder, social anxiety disorder, post-traumatic stress syndrome, obsessive-compulsive disorder and related disorders of impulse control. The methods also find use in treating major depression and severe, vital or “melancholic” depression. Psychiatric disorders, including depression and anxiety disorders, amenable to treatment by reducing, inhibiting or preventing α-2,8-sialyltransferase activity are reviewed in Goodman and Gilman's The Pharmacological Basis of Therapeutics, supra.

In therapeutic applications, the α-2,8-sialyltransferase inhibitors of the invention are administered to an individual already suffering from an inappropriate or undesirable psychiatric disorder, including depression and/or anxiety disorders. Compositions that contain α-2,8-sialyltransferase inhibitors or agents that bind to and block polysialic acid moieties on cell types or tissue types of interest are administered to a patient in an amount sufficient to suppress the undesirable psychiatric disorder and to eliminate or at least partially arrest symptoms and/or complications. An amount adequate to accomplish this is defined as “therapeutically effective dose.” Amounts effective for this use will depend on, e.g., the inhibitor composition, the manner of administration, the stage and severity of the disease being treated, the weight and general state of health of the patient, and the judgment of the prescribing physician. Inhibitors of α-2,8-sialyltransferase activity can be administered chronically or acutely to treat a particular psychiatric disorder. In certain instances, it will be appropriate to administer an inhibitor of α-2,8-sialyltransferase activity prophylactically, for instance in subjects with a history of psychiatric disorders, including depression and fear or anxiety disorders.

Alternatively, DNA or RNA that inhibits expression of one or more sequences encoding an α-2,8-sialyltransferase, such as an antisense nucleic acid, a small-interfering nucleic acid (i.e., siRNA) or a nucleic acid that encodes a peptide that blocks expression or activity of a α-2,8-sialyltransferase can be introduced into patients to achieve inhibition. U.S. Pat. No. 5,580,859 describes the use of injection of naked nucleic acids into cells to obtain expression of the genes which the nucleic acids encode.

Therapeutically effective amounts of an α-2,8-sialyltransferase inhibitor or enhancer compositions of the present invention generally range for the initial administration (that is for therapeutic or prophylactic administration) from about 1.0 mg to about 10 g of α-2,8-sialyltransferase inhibitor for a 70 kg patient, usually from about 10 mg to about 5 g, and preferably between about 2 mg and about 1 g. These doses can be followed by repeated administrations over weeks to months depending upon the patient's response and condition by evaluating symptoms of anxiety, fear and/or depression.

It must be kept in mind that the compositions of the present invention may be employed in severe and/or acute disease states, that is, life-threatening or potentially life threatening situations. In such cases, in view of the minimization of extraneous substances and the relative nontoxic nature of the inhibitors, it is possible and may be felt desirable by the treating physician to administer substantial excesses of these compositions.

For prophylactic use, administration should be given to subjects at risk or those with a history of psychiatric disorders characterized by anxiety, fear or depression. Therapeutic administration may begin at the first sign of disease or the detection or shortly after diagnosis of the psychiatric disorder. This is often followed by repeated administration until at least symptoms are substantially abated and for a period thereafter.

The pharmaceutical compositions for therapeutic or prophylactic treatment are intended for parenteral, topical, oral or local administration. Preferably, the compositions are formulated for oral administration. In certain embodiments, the pharmaceutical compositions are administered parenterally, e.g., intravenously, subcutaneously, intradermally, or intramuscularly. Compositions of the invention are also suitable for oral administration. Thus, the invention provides compositions for parenteral administration which comprise a solution of the α-2,8-sialyltransferase inhibiting agent dissolved or suspended in an acceptable carrier, preferably an aqueous carrier. A variety of aqueous carriers may be used, e.g., water, buffered water, 0.9% saline, 0.3% glycine or another suitable amino acid, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of α-2,8-sialyltransferase inhibiting agents of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The α-2,8-sialyltransferase inhibitors of the invention may also be administered via liposomes, which serve to target the conjugates to a particular tissue, such as a neural progenitor tissue, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among neuronal progenitor cells, or with other therapeutic compositions. Thus, liposomes filled with a desired peptide, small molecule or conjugate of the invention can be directed to the site of, for example, neural progenitor cells, where the liposomes then deliver the selected α-2,8-sialyltransferase inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to neuronal progenitor cells, a ligand to be incorporated into the liposome can include, e.g., antibodies or fragments thereof specific for cell surface determinants of the desired neuronal progenitor cells. A liposome suspension containing a peptide or conjugate may be administered intravenously, locally, topically, etc. in a dose which varies according to, inter alia, the manner of administration, the conjugate being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more conjugates of the invention, and more preferably at a concentration of 25%-75%.

For aerosol administration, the inhibitors are preferably supplied in a suitable form along with a surfactant and propellant. Typical percentages of α-2,8-sialyltransferase inhibitors are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

The present invention also provides methods of monitoring psychiatric disorders characterized by anxiety, fear or depression by detecting the levels of polysialic acid moieties in a sample from a patient. This can be performed according to standard methods for detection of desired carbohydrate structures. Detection moieties that bind to, for example, polysialic acid, or to the acceptor substrate for the polysialic acid, are used to detect whether the polysialic acid is present in the sample.

In typical embodiments, the detection moieties are labeled with a detectable label. The detectable labels can be primary labels (where the label comprises an element that is detected directly or that produces a directly detectable element) or secondary labels (where the detected label binds to a primary label, as is common in immunological labeling). An introduction to labels, labeling procedures and detection of labels is found in Polak and Van Noorden (1997) Introduction to Immunocytochemistry, 2nd ed., Springer Verlag, NY and in Haugland (2002) Handbook of Fluorescent Probes and Research Products, 9th Edition, a combined handbook and catalogue published by Molecular Probes, Inc., Eugene, Oreg., available in updated form on their worldwide website at probes.com. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocyanate (TRITC), etc.), digoxigenin, biotin, phycoerythrin, AMCA, CyDyes, and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectral colorimetric labels such as colloidal gold or colored glass or plastic (e.g. polystyrene, polypropylene, latex, etc.) beads. The label may be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

Preferred labels include those that use: 1) chemiluminescence (for example, using horseradish peroxidase or luciferase) with substrates that produce photons as breakdown products as described above) 2) color production (for example, using both horseradish peroxidase and/or alkaline phosphatase with substrates that produce a colored precipitate); 3) hemifluorescence using, e.g., alkaline phosphatase and an alkaline phosphatase substrate or other substrates that produce fluorescent products, 4) fluorescence (e.g., using Cy-5, (available from Amersham Biosciences), fluorescein, and other fluorescent tags); or 5) radioactivity. Kits for labeling and detection using the foregoing labeling techniques are available from, for example, Molecular Probes (Eugene, Oreg.), Amersham Biosciences (Piscataway, N.J.), Roche Applied Science (Indianapolis, Ind.), and Invitrogen Corp. (Carlsbad, Calif.). Other methods for labeling and detection will be readily apparent to one skilled in the art.

Preferred enzymes that can be conjugated to detection reagents of the invention include, e.g., luciferase, and horse radish peroxidase. The chemiluminescent substrate for luciferase is luciferase. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer; and 3,3,5,5′-tetramethylbenzidine (TMB), 3,3′diaminobenzidine (DAB), 3-amino-9-ethylcarbazole (AEC), and 4-chloro-1-napthol (4ClN), which are detected visually. Other suitable substrates are known to those skilled in the art.

In general, a detector which monitors a particular label is used to detect the label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling moieties is digitized for subsequent computer analysis.

Commercially available detection moieties that are suitable for use in the methods of the invention include SNA-fluorescein isothiocyanate (FITC) lectin (FL-1301, Vector Laboratories, Burlingame CA) and biotinylated SNA lectin (B-1305, Vector Laboratories) for α2,3 sialyl galactosides. For detection of α2,6 sialylgalactosides, MAL II-FITC lectin and biotinylated MAL II lectin (3-1265, Vector Laboratories) are examples of suitable detection moieties.

An effective anxiolytic treatment is indicated by a decrease in behaviors associated with anxiety, fear or depression, as measured according to a clinician (i.e., a psychiatrist) to by the patient. Alternatively, methods for detecting levels of specific α-2,8-sialyltransferase activities can be used. Standard assays for detecting α-2,8-sialyltransferases such as ST8Sia-II sialyltransferases are described herein. Again, an effective anxiolytic or anti-depressive treatment is indicated by a substantial reduction in activity of the particular α-2,8-sialyltransferase. As used herein, a “substantial reduction” in the appropriate polysialic acid levels or α-2,8-sialyltransferase activity refers to a reduction of at least about 30% in the test sample compared to an untreated control. Preferably, the reduction will be at least about 50%, more preferably at least about 75%, and most preferably polysialic acid or α-2,8-sialyltransferase activity levels will be reduced by at least about 90% in a sample from a treated mammal compared to an untreated control.

IV. Transgenic Animals that Lack ST8Sia-II sialyltransferase

The invention also provides chimeric and transgenic nonhuman animals which contain cells that lack at least one ST8Sia-II gene that is found in wild-type cells of the animal, and methods for producing such animals. These animals are useful for several purposes, including the study of the mechanisms by which polysialic acid influences anxiety and fear responses and other effects. Such “knockout” animals can also be used for producing glycoproteins and glycolipids that, when produced in a wild-type animal, would carry a sialic acid residue that is not desirable for a particular application.

A “chimeric animal” includes some cells that lack the functional ST8Sia-II gene and other cells that do not have the inactivated gene. A “transgenic animal,” in contrast, is made up of cells that have all incorporated the specific modification which renders the ST8Sia-II gene inactive. While a transgenic animal is capable of transmitting the inactivated ST8Sia-II gene to its progeny, the ability of a chimeric animal to transmit the mutation depends upon whether the inactivated gene is present in the animal's germ cells. The modifications that inactivate the ST8Sia-II gene can include, for example, insertions, deletions, or substitutions of one or more nucleotides. The modifications can interfere with transcription of the gene itself, with translation and/or stability of the resulting mRNA, or can cause the gene to encode an inactive ST8Sia-II polypeptide.

The present methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Pinkert, Calif., Ed., Transgenic Animal Technology: A Laboratory Handbook, 2^{nd} Edition, Academic Press, 2003; and Houdebine, Animal Transgenesis and Cloning, John Wiley & Sons, 2003.

One method of obtaining a transgenic or chimeric animal having an inactivated ST8Sia-II gene in its genome is to contact fertilized oocytes with a vector that includes a ST8Sia-II-encoding polynucleotide that is modified to contain an inactivating modification. For some animals, such as mice, fertilization is performed in vivo and fertilized ova are surgically removed. In other animals, particularly bovines, it is preferably to remove ova from live or slaughterhouse animals and fertilize the ova in vitro. See DeBoer et al., WO 91/08216. In vitro fertilization permits the modifications to be introduced into substantially synchronous cells. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoel cavity, typically at the 64 cell stage. If desired, the presence of a desired inactivated ST8Sia-II gene in the embryo cells can be detected by methods known to those of skill in the art. Methods for culturing fertilized oocytes to the pre-implantation stage are described by Gordon et al. (1984) Methods Enzymol. 101: 414; Hogan et al. Manipulation of the Mouse Embryo: A Laboratory Manual, C.S.H.L. N.Y. (1986) (mouse embryo); Hammer et al. (1985) Nature 315: 680 (rabbit and porcine embryos); Gandolfi et al. (1987) J. Reprod. Fert. 81: 23-28; Rexroad et al. (1988) J. Anim. Sci. 66: 947-953 (ovine embryos) and Eyestone et al. (1989) J. Reprod. Fert. 85: 715-720; Camous et al. (1984) J. Reprod. Fert. 72: 779-785; and Heyman et al. (1987) Theriogenology 27: 5968 (bovine embryos). Sometimes pre-implantation embryos are stored frozen for a period pending implantation. Pre-implantation embryos are transferred to an appropriate female resulting in the birth of a transgenic or chimeric animal depending upon the stage of development when the transgene is integrated. Chimeric mammals can be bred to form true germline transgenic animals.

Alternatively, the modified ST8Sia-II gene can be introduced into embryonic stem cells (ES). These cells are obtained from preimplantation embryos cultured in vitro. See, e.g., Hooper, ML, Embryonal Stem Cells: Introducing Planned Changes into the Animal Germline (Modern Genetics, v. 1), Int'l. Pub. Distrib., Inc., 1993; Bradley et al. (1984) Nature 309, 255-258. Transformed ES cells are combined with blastocysts from a non-human animal. The ES cells colonize the embryo and in some embryos form the germ line of the resulting chimeric animal. See, Jaenisch (1988) Science 240: 1468-1474. Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al. (1997) Nature 385: 810-813.

The introduction of the modified ST8Sia-II gene into recipient cells can be accomplished by methods known to those of skill in the art. For example, the modified gene can be targeted to the wild type sialyltransferase locus by homologous recombination. Alternatively, a recombinase system can be employed to delete all or a portion of a locus of interest. Examples of recombinase systems include, the cre/lox system of bacteriophage P1 (see, e.g., Gu et al. (1994) Science 265: 103-106; Terry et al. (1997) Transgenic Res. 6: 349-356) and the FLP/FRT site specific integration system (see, e.g., Dymecki (1996) Proc. Nat'l Acad. Sci. USA 93: 6191-6196). In these systems, sites recognized by the particular recombinase are typically introduced into the genome at a position flanking the portion of the gene that is to be deleted. Introduction of the recombinase into the cells then catalyzes recombination which deletes from the genome the polynucleotide sequence that is flanked by the recombination sites. If desired, one can obtain animals in which only certain cell types lack the sialyltransferase gene of interest. See, e.g., Tsien et al. (1996) Cell 87: 1317-26; Brocard et al. (1996) Proc. Nat'l. Acad. Sci. USA 93: 10887-10890; Wang et al. (1996) Proc. Nat'l. Acad. Sci. USA 93: 3932-6; Meyers et al. (1998) Nat. Genet. 18: 136-41).

### EXAMPLES

The following examples are offered to illustrate, but not to limit the claimed invention.

### Example 1

Materials and Methods

Mutagenesis of the Mouse ST8Sia-II Gene

A region of the murine ST8Sia-II allele was isolated from a mouse129/SvJ genomic DNA library and used to construct a targeting vector. An EcoRI-XhoI fragment containing exon was flanked by two loxP sequences as described (Marth, J. Clin. Invest. (1996) 97:1999-2002). Targeted ES clones were identified by polymerase chain reaction, and characterized by genomic Southern blotting. Multiple ES clones bearing the systemic (type I) deletion of the targeted ST8Sia-II allele were obtained after transient expression of Cre recombinase. Targeted ES cells were injected into C57BL/6 blastocysts to generate chimeric mice. Mutant ST8Sia-II alleles were maintained on the C57BL/6 background for more than 6 generations prior to mouse phenotype analysis.

RNA analyses

Total RNA was purified from brains at different ages by using TRIZOL® solution (Invitrogen) and used for Northern hybridization and RT-PCR as described previously (Ong et al., Glycobiology (1998) 8:415-424). RT-PCR was performed using cDNAs synthesized with oligo(dT) as a primer by Superscript II reverse transcriptase (Invitrogen) and 10 pmol of primers set for murine ST8Sia-II (mX) or ST8Sia-IV (mP). The following primers were used:

mX-Tg1: 5′-CTGGAGGCAGAGGTACAATCAGATC-3′
(nucleotides 104-128)
and
mX-Tg2: 5′-CCTCAAAGGCCCGCTGGATGACAGA-3′
(nucleotides 646-622);
mP-Tg1: 5′-AGGCTGGCTCCACCATCTTCCAACA-3′
(nucleotides 173-197)
and
mP-Tg2: 5′-CTCTGTCACTCTCATTCCGAAAGCC-3′
(nucleotides 625-601).

Western Blot Analysis

After brains were removed, various brain regions were dissected and homogenized with RIPA buffer (150 mM NaCl; 50 mM Tris-HCl, pH 7.4; 1% NP40; 0.1% SDS; 5 mM EDTA) containing proteinase inhibitor cocktail (Roche Applied Science). Equivalent amounts of proteins were loaded onto a 5% SDS-PAGE gel, and transferred onto PVDF membranes (Millipore). Some tissue extracts were incubated with endo-N (Hallenbeck et al., J. Biol. Chem. (1987) 262:3553-3561) for 1 hour at 37° C. prior to loading. Membranes were blocked with 10% dry milk in 20 mM Tris-buffered saline, pH 7.6, containing 0.1% Tween-20 (TBST), incubated with either mouse anti-PSA 5A5 (Developmental Studies Hybridoma Bank, diluted 1:1000 in TBST) or rat anti-NCAM H28 (Immunotech, diluted 1:200), and followed by peroxidase-conjugated antimouse IgM (1:4000) or anti-rat IgG (1:3000), respectively to detect by ECL kit (Amersham Biosciences).

### 5-Bromo-2′-deoxyuridine (BrdU)-Labeling

BrdU (20 mg/ml in 0.007N NaOH and 0.9% NaCl) was injected (intraperitoneal) into the ST8Sia-II-deficient and wild-type mice (50 mg/kg body weight) for 5 times at 2 hour intervals over an 8 hour period. One week after the last administration, the mice were deeply anesthetized with Avertin (0.015 ml/g body weight) and brains were removed. The brains were fixed with Carnoy (60% ethanol, 30% chloroform, 10% acetic acid) and embedded in paraffin to cut sagittal or coronal sections at 10 μm. Every third section was collected and 10 sections, which cover 300 μm in the center of the hippocampus, were stained for BrdU. BrdU-positive cells were detected by BrdU specific monoclonal antibody (Roche Applied Science) and ALEXA FLUOR® 488 goat anti-mouse IgG1 (Molecular Probes). To measure distribution of embryonic neural stem cells in brains, BrdU was administrated into pregnant heterozygous mice (100 mg/kg at embryonic day 16, E16) that were mated with heterozygous male mice. Brains from neonatal mice and 10 days postnatal mice were fixed and stained as described above.

Histology

To stain sections with SMI-32 (Stemberger Monoclonal), Tuj1 (BAbCo), and antibodies for synapsin I (Chemicon), GFAP or MAP2 (Roche Applied Science), sections were prepared as described for BrdU detection. To stain sections with anti-PSA 12F8 (BD Biosciences), anti-NCAM H28, and anti-calbindine D-28K (Chemicon), mice were perfused intracardially with phosphate-buffered saline (PBS) followed by 4% paraformaldehyde in 0.1 M phosphate buffer, pH 7.4. Sagittal or coronal sections frozen in OCT compound (Sakura Finetek) were cut at 30 μm by a cryostat and collected onto PBS. The sections were treated with 0.3% H₂O₂ for 5 min, and incubated with PBS containing 0.1% normal goat serum and 0.25% Triton-X 100. Sections were then treated with primary antibodies in the above blocking solution, followed by appropriate biotinylated secondary antibodies (Zymed and Vector Laboratories). The sections were treated with ABC reagent and DAB substrate (Vector Laboratories), transferred onto slides, and counterstained with cresyl violet. For immunofluorescence staining, tissues were incubated with primary antibodies in 1% normal goat serum and followed by ALEXA FLUOR® 488 or 594 conjugated goat anti-rabbit IgG, -rat IgM, -mouse IgG1, and -mouse IgG2a (Molecular Probe). Then the sections were reacted with Hoechst 33342 (Sigma) in PBS before mounting. With some slides, Nissl staining was performed with NEUROTRACER™ Fluorescent Nissl Stains (Molecular Probe).

For Timm's staining, mice were perfused intracardially first with 15-50 ml of 0.37% sodium sulfide solution, followed by 4% paraformaldehyde in PBS to prepare frozen sections as described above. The sections were incubated in darkness for 40 min in a solution containing 30% gum arabic, 2.55% citric acid, 2.35% sodium citrate, 1.7% hydroquinone, and 0.1% silver nitrate. The slides were then rinsed in running water for 10 minutes and counterstained with cresyl violet.

Electrophysiology

Hippocampal slices from five- to six-month-old ST8Sia-II-deficient mice and their wild-type littermates were used for recordings. After halothane anesthesia, decapitation and removal of the brain, the hippocampi were cut with a VT 1000M vibratome (Leica, Nussloch, Germany) in slices 300 μm thick in ice-cold artificial cerebrospinal fluid (ACSF) containing (in mM): 250 sucrose, 25 NaHCO₃, 25 glucose, 2.5 KCl, 1.25 NaH₂PO₄, 2 CaCl₂, and 1.5 MgCl₂, pH 7.3. The slices were then kept at room temperature in a chamber filled with carbogen-bubbled ACSF, containing 125 mM NaCl instead of 250 mM sucrose, for at least 2 hr before the start of recordings (modified from Edwards et al., J. Physiol. (1990) 430:213-249). In the recording chamber, slices were continuously superfused with carbogen-bubbled ACSF (2-3 ml/min) at room temperature.

For recordings in the CA3 region the slices were prepared as for recordings in the CA1 region, but with some modifications. Before decapitation, mice were transcardially perfused with ice-cold ACSF, containing (in mM): 250 sucrose, 25 NaHCO₃, 25 glucose, 2.5 KCl, 1.25 NaH₂PO₄, 0.5 CaCl₂, and 6 MgCl₂, pH 7.3. Slices were cut according to Claiborne and colleagues (Hippocampus (1993) 3:115-121). Exchange of sucrose-containing ACSF with normal (containing 2.5 mM CaCl₂ and 1.5 mM MgCl₂) was performed gradually using peristaltic pumps.

Schaffer collateral-CA1 extracellular recordings of focal fEPSPs were obtained from the stratum radiatum of the CA1 region in response to stimulation of Schaffer collaterals by an electrode placed approximately 400 μm apart from the recording electrode in the stratum radiatum of the CA1 region. Recordings and stimulations were performed with glass pipettes filled with ACSF having a resistance of 2 MΩ. Basal synaptic transmission was monitored at 0.033 Hz. The slices were maintained at room temperature.

Homosynaptic LTP in the CA1 region was induced by theta-burst stimulation (TBS) applied orthodromically to Schaffer collaterals and recorded extracellularly in the stratum radiatum. A TBS consisted of 10 bursts delivered at 5 Hz. Each burst consisted of 4 pulses delivered at 100 Hz. Duration of pulses was 0.2 ms, and five TBSs were applied every 20 s to induce LTP (Eckhardt et al., J. Neurosci. (2000) 20:5234-5244). The stimulation strength was in the range of 40-70 μA to provide fEPSPs with an amplitude of 50% of the subthreshold maximum. The mean slope of fEPSPs recorded 0-10 min before TBS was taken as 100%. The transient potentiation immediately following TBS (or STP, short-term potentiation) was measured as a maximal increase in the fEPSPs slope during 1 min after LTP induction. The values of LTP were calculated as increase in the mean slopes of fEPSPs measured 50-60 min after TBS.

Mossy fiber-CA3 extracellular recordings and stimulations were both performed with glass pipettes filled with ACSF and having a resistance of 2 MΩ with stimulation strength of approximately 40 μA. The stimulating electrode was placed close to the internal side of the granule cell layer. The recording pipette was placed in the stratum lucidum of the CA3 region. The mossy fiber responses selected for recording were of 40-60 μV, with a fast rise-time and decay of fEPSPs (total duration of fEPSP<10 ms, rise time <3.5 ms), large paired pulse facilitation (>170%), and prominent frequency facilitation (>200%). The selected responses had no hallmarks of polysynaptic activation, such as jagged decay phase with multiple peaks, or variable latencies of fEPSPs.

The LTP-inducing high-frequency stimulation (HFS) consisted of one train of stimuli applied at 100 Hz during 1 sec once (“weak” stimulation protocol) or repeated four times with an interval of 20 sec (“strong” stimulation protocol). To evoke LTP exclusively in mossy fiber synapses, which are known to undergo LTP in a NMDA receptor-independent manner, the NMDA receptor antagonist AP-5 (50 μM; Tocris, Bristol, UK) was applied 15 minutes before and during HFS. All recorded mossy fiber responses followed presynaptic stimulation of 100 Hz and showed no changes in the shape of responses after induction of LTP. To additionally confirm that the fEPSPs recorded were evoked by the stimulation of mossy fibers and not by the associational/commissural pathway, an agonist of type II metabotropic glutamate receptors (L-CCG1, 10 μM; Tocris), which is known to reduce synaptic transmission in CA3 mossy fiber synapses (Manzoni, et al. Neuropharmacology (1995) 34:965-71), was applied at the end of each experiment. Slices in which responses were reduced by at least 70% were selected for analysis. Basal synaptic transmission was monitored at 0.033 Hz. The mean amplitude of fEPSPs recorded 0-10 min before HFS was taken as 100%. Post-tetanic potentiation (PTP) was calculated as the maximal increase in the amplitude of fEPSP after HFS. The values of LTP were calculated as increase in the mean amplitude of fEPSPs measured 50-60 min after HFS.

Data acquisition and measurements were performed using an EPC-9 amplifier and Pulse software (Heka Elektronik, Lambrecht/Pfalz, Germany). Values in electrophysiological experiments are reported as mean±SEM (standard error of the mean). Student's t-test was used to assess statistical significance using Sigma Plot 5.0 software (SPSS, Chicago, Ill., USA).

Nerve Tract Tracing Between Hippocampus and Amygdala

Neuronal tracing between hippocampus and amygdala was analyzed in eight mice of each genotype by injecting biotinylated dextran amine (BDA, Molecular Probes) into the amygdala. After the mouse was deeply anesthetized with ketamine (1.25 mg/g) and xylazine (0.07 mg/g), BDA (100% in 0.1 M phosphate buffer, pH 7.4; 0.2 μL) was stereotaxically injected into amygdala by using Picospritzer (Parker Instrumentation) and the coordinates (A: −0.12, L: −0.27, D: −0.4 in cm from Bregma; Franklin and Paxinos, The mouse brain in stereotaxic coordinates, 1996, San Diego: Academic Press). The animals were allowed to recover under close observation and returned to their cage. Six days after injection, brains were fixed as described above and cryosections were analyzed immunohistochemically using FITC-labeled Avidin (Vector Laboratories).

Metabolic and Behavioral Parameters

Two separate cohorts of four month-old male mice were analyzed. The first cohort of mice consisted of 9 ST8Sia-II deficient (Δ/Δ) mice and 10 wild-type (wt/wt) littermate controls. These were assessed in a behavioral test battery modified from that used by McIlwain et al. Physiol. Behav. (2001) 73:705-717) and described in detail previously (Corbo et al., J. Neurosci. (2002) 22:7548-7557), and included parameters such as metabolic performance, physical appearance, sensorimotor reflexes, motor activity, nociception, acoustic startle, sensorimotor gating, and assessments of learning and memory. Concern that testing mice in such a large battery could influence the behavior in any individual task, and that multiple assessments increases the probability of a Type I statistical error, a second cohort of mice (wt/wt: n=16; Δ/Δ: n=15) was analyzed in fear conditioning tasks and the open field test. In addition, a subset of this second cohort (wt/wt: n=12; Δ/Δ: n=9) was subsequently analyzed in the water maze (a task not run with the first cohort) and some of these mice (wt/wt n=8; Δ/Δ n=8) were also assessed for metabolic measures.

In the open field test, exploratory locomotor activity was measured in a 30 minutes test period in an area of 45×45 cm using a Digiscan apparatus (Accuscan Electronics, Columbus, Ohio). Vertical activity (rearing) total distance (cm), and center distance were recorded. The center distance divided by the total distance traveled is an indicator of anxiety-related behavior.

The water maze task constituted a pre-training phase during which all mice were tested for two days in a straight-swim pre-training protocol. Mice received 16 trials (8 trials over two days) in a 31×60 cm rectangular tank that was located in a different room than the circular tank used in the hidden platform trials. The platform was located 1 cm below the water opposite from the start location. Latency to climb onto the platform was the dependent measure. Criteria for advancing to the hidden platform trials was completing 6 of 8 trials under 10 seconds on the second day. This pretraining procedure provided experience with swimming and climbing onto a submerged platform without exposing the mice to the spatial cues used in the hidden platform trials. This procedure both screens for mice with severe motor deficits and reduces behavioral variability often seen on the first day hidden platform testing. All mice successfully passed this pretraining phase. Hidden platform testing followed in which extra-maze visual cues were hung from a curtain located around a 1.26 meter diameter circular tank. The water was made opaque with the addition of non-toxic paint. The 10 cm diameter escape platform was located 1 cm below the surface of the water and a Polytrack video-tracking system (San Diego Instruments) was used to collect mouse movement data (location, distance and latency) during training and probe trials. Each mouse was given 8 trials a day, in 2 blocks of 4 trials for four consecutive days. After 36 trials, each animal was given a 60 sec probe trial. During the probe test, the platform was removed and quadrant search times were measured. Visual cue testing was performed 1 day after the last hidden-platform training trial, wherein mice were trained to locate a visible-cued platform. The visible cue was a gray plastic cube (9 cm) attached to a pole such that it was 10 cm above the platform. On each trial of the visible platform test, the platform was randomly located in one of the four quadrants. Mice were given 8 trials, in blocks of four trials, and the latency to find the platform was recorded for each trial.

Fear conditioning tests involved chambers (26×22×18 cm high) made of clear Plexiglas were placed in a 2×2 array (Med Associates). A video camera was connected to a video-based system for digital recording and subsequent analysis of freezing behavior upon mild electric shock (FreezeFrame, Actimetrics, St. Evanston, Ill.). The conditioned stimulus (CS) was an 85 db 2,800 Hz 20-second tone and the unconditioned stimulus (US) was a scrambled foot shock at 0.75 mA presented during the last 3 seconds of the CS. Mice were placed in the test chamber for 3 minutes before the CS and freezing behavior was recorded. Freezing was defined as the absence of movement other than breathing and thresholds were selected via the software of high correlation with human observers. Three CS/US pairings are given with 1 minute spacing and freezing during the CS was also recorded. Twenty-four hours later each mouse was placed back into the shock chamber and freezing response was recorded for 3 minutes (context test). Two hours later, the chambers were modified to present a different environmental context (e.g. shape, odor, color changes) and the mouse was placed in this novel environment. Freezing behavior was recorded for 3 minutes before and during three CS presentations (cued conditioning). The time spent freezing was converted to a percent value.

For the passive avoidance analysis, a two-compartment light/dark apparatus (35×18×30 cm, Coulbourn Instruments, Allentown, Pa.) was used. Each mouse was placed individually in the lighted compartment. When the animal entered the dark compartment, a guillotine door closed behind the mouse and a foot shock of 0.4 mA was delivered through the grid floor of the dark compartment for 3 seconds. If the mouse did not enter the dark compartment within 10 minutes, it was excluded from the retrieval test. In the retrieval trial performed 24 hours later, the latency for the mice to enter the dark compartment was recorded. The maximum latency was 600 seconds.

Metabolic chambers termed CLAMS (Comprehensive Lab Animal Monitoring System; Columbus Instruments, Columbus, Ohio) automatically recorded metabolic parameters including volume of carbon dioxide produced (VCO₂), volume of oxygen consumed (VO₂), respiration (RER=VCO₂/VO₂), and caloric (heat) value ((3.815+1.232×RER)×VO₂)), motion in all three axes in time, and consumption of food and water. Data was collected every 30 minutes over three 12 hour dark cycles and two 12 hour light cycles and analyzed as mean values over each 12 hour period with the exception of food and water intake which were added to the total during subsequent cycles.

Pulmonary function was scored by measurement of the uptake of carbon monoxide (CO). A carbon monoxide uptake monitor (Columbus Instruments, Columbus, Ohio) measured the CO level in a sealed chamber after exposing the mouse to a 60 second interval of air with 0.17% CO. The mean breath per minute was also recorded. Each animal was tested once.

Blood pressure was determined by a non-invasive blood pressure tail-cuff system (Columbus Instruments) that measures systolic blood pressure in addition to heart rate and relative changes in diastolic and mean blood pressure. Individual mice were placed in a small cylinder chamber, occlusion and sensor cuffs were placed on the tail, and the tail was warmed to 37° C. Mice were first acclimated to the restraining chamber, tail cuffs, and the heat fan for 30 minutes for two days prior to testing. The mean of 4 measurements on the third day is reported and analyzed by the Student t-test.

Results

ST8Sia-II Deficiency in the Mouse Germline

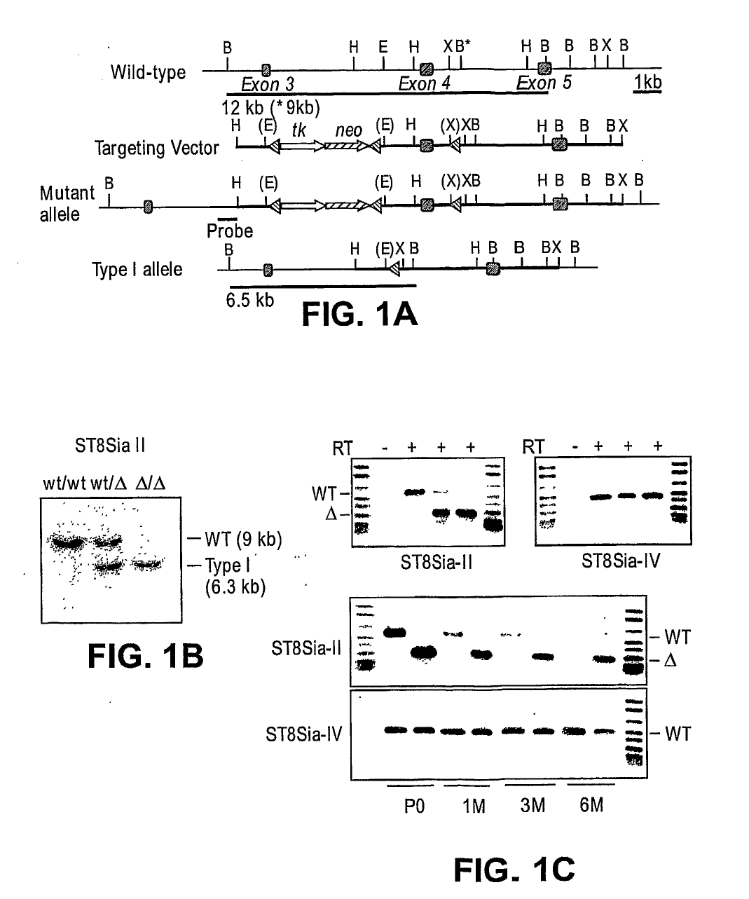

The ST8Sia-II gene is highly conserved among mammals and includes multiple exons that divide the catalytic domain (Yoshida et al., J. Biol. Chem. (1996) 271:30167-30173). Exon 4 encodes a significant portion of the sialylmotif L, a peptide region that takes part in donor substrate recognition and is essential for sialyltransferase activity (Datta and Paulson, J. Biol. Chem. (1995) 270:1497-1500). DNA encoding exon 4 of the ST8Sia-II gene was targeted for elimination from the mouse genome (FIG. 1a). Targeted ES cells were transfected with Cre recombinase to delete loxP-flanked exon 4 sequence, and ES clones that bore the type I deletion were selected for Southern hybridization (FIG. 1b). Type I ES cells were identified and injected into C57BL/6 blastocysts to obtain chimeric mice. Subsequently the mutant allele was transmitted into germline and bred into the C57BL/6 strain. Mice homozygous for the type I (deleted,) ST8Sia-II allele appeared to have no significant abnormality in body weight or brain size up to 6 months of age, in spite of the fact that high level expression of ST8Sia-II occurs in wild-type embryos at early developmental stages. Breeding from heterozygous males and females produced mice with genotypes close to Mendelian ratios (wt/wt:wt/Δ: Δ/Δ=29:50:21%; n=255), implying that the mutated ST8Sia-II allele was not deleterious in mouse development.

To confirm disruption of ST8Sia-II at mRNA level, Northern hybridization and RTPCR were performed. These analyses revealed that the truncated form of ST8Sia-II mRNA lacking exon 4 and an intact sialyl motif is present in both heterozygous and the ST8Sia II null mice (FIG. 1c). The sequence of the mutant ST8Sia-II mRNA indicated that a truncated form of ST8Sia-II protein lacking exon 4 might be translated by in-frame splicing of exon 3 to exon 5. However, the truncated ST8Sia-II protein was shown to lack polysialyltransferase activity by immunofluorescence staining with PSA antibodies of the cells transfected with the mutant ST8Sia-II cDNA (data not shown). Northern hybridization and RT-PCR also demonstrated that the disruption of ST8Sia-II does not affect the level of ST8Sia-IV mRNA in ST8Sia-II deficient mice (FIG. 1c).

Regional Defects in Brain Polysialic Acid Expression

Histological examination of macroscopic brain structure indicated normal development in the absence of ST8Sia-II activity (data not shown). Although NCAM-deficient mice have a smaller olfactory bulb and thicker rostral migratory stream (RMS) (Tomasiewicz et al., Neuron (1993) 11:1163-1174; Cremer et al., Nature (1994) 367:455-459; Hu et al., Neuron (1996) 16:735-743), ST8Sia-II deficient mice bear a normal size olfactory bulb and RMS (data not shown), as was also observed in ST8Sia-IV deficient mice (Eckhardt et al., J. Neurosci. (2000) 20:5234-5244).

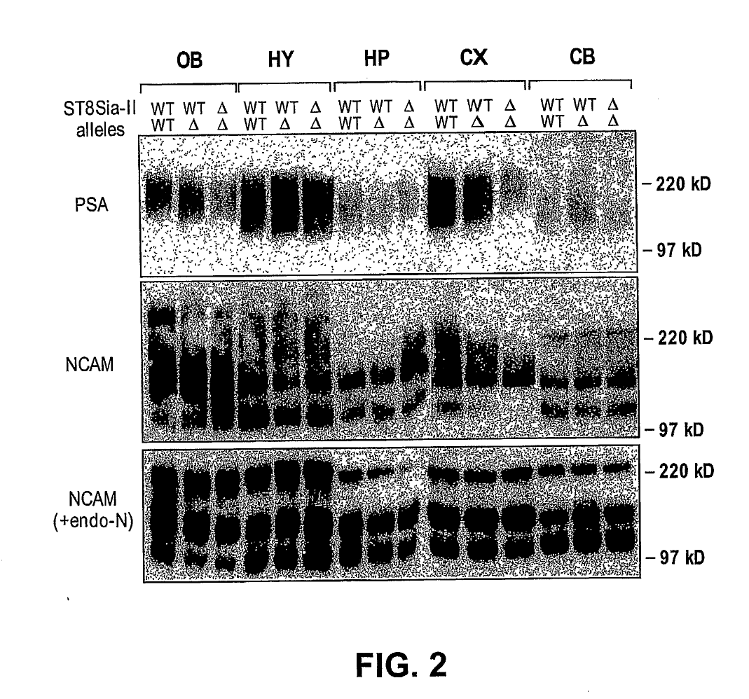

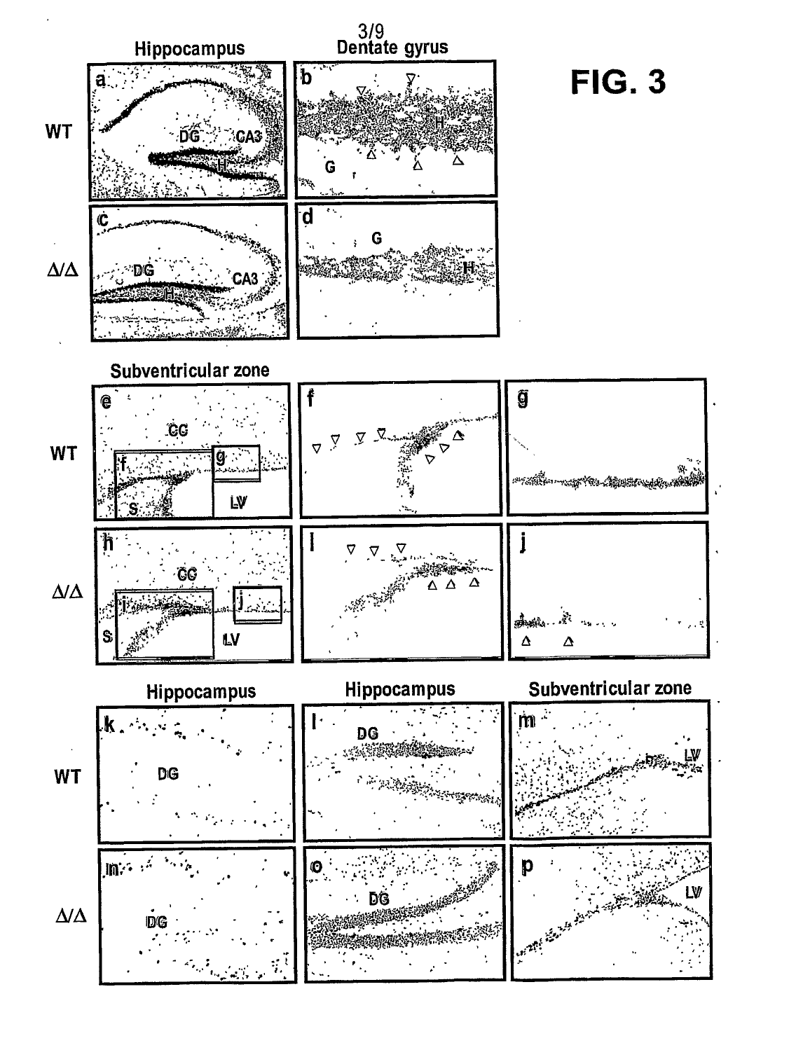

PSA expression in ST8Sia-II deficient mice was altered in comparison with wild-type littermates, being reduced though not eliminated in the olfactory bulb and cerebral cortex of 8-week-old adult mice (FIG. 2). There was no apparent quantitative alteration of PSA levels in the hypothalamus, hippocampus, and cerebellar cortex. A closer examination of the hippocampus revealed an altered pattern of PSA expression among ST8Sia-II deficient mice (FIGS. 3a, d). The hippocampus is one of the regions that produce PSA in adults, and PSA is normally highly expressed in granule cells, hilus, and mossy fibers. Neurogenesis in the dentate gyrus takes place throughout adulthood and neuronal progenitor cells are located at the border between the hilus and the granule cell layer. Newly generated cells migrate into the granule cell layer and normally express high levels of PSA. In the four groups of wildtype mice examined at different ages, progenitor cells bearing PSA decreased with increased age as expected (Kuhn et al., J. Neurosci. (1996) 16:2027-2033). Among ST8Sia-II deficient littermates, the number of granule cells expressing PSA was markedly reduced and in some cases none were detected (FIGS. 3b, d).

The subventricular zone (SVZ) is another area of neural stem cell genesis in the adult brain. Among wild-type mice, PSA is easily detectable in the anterior region of the SVZ (SVZa), likely indicating migrating neuroblasts, and is positive in the region of the striatum. Among ST8Sia-II deficient mice, PSA is also detectable in the SVZa and the striatum (FIGS. 3e, f, h, i). Levels of PSA in the migratory pathway and the chain migration pattern of precursor cells appeared to be unchanged (FIGS. 3f, i), consistent with results of staining by GFAP and β-tubulin antibodies (data not shown). However, in contrast to wild-type littermates, PSA was greatly reduced among newly generated neural cells along the lateral ventricle (FIGS. 3g, j). These results indicate that PSA on neural precursor cells in the SVZ is synthesized primarily by the ST8Sia-II polysialyltransferase.

Deficiency of PSA formation in the context of neurogenesis was further examined by BrdU-labeling of neural precursors. At embryonic day 16, these cells are distributed in the hippocampus as well as the cortex. The labeled cell number and migration pattern in the dentate gyrus and the pyramidal cell layer were not distinguishable between wild-type and ST8Sia-II deficient mice (FIGS. 3k, n). Even in adult, BrdU-positive mitotic cells were found in dentate gyrus and SVZ of both genotypes (FIGS. 31, m, o, p). The number of BrdU-positive cells in the adult dentate gyrus of ST8Sia-II deficient mice was not significantly lower than that of wild-type mice (wt/wt: 10.9±2.9, n=4; Δ/Δ: 8.7±2.0, n=4; P>0.05). These findings support the view that PSA formation in neural progenitor cells is provided primarily by ST8Sia-II, and show that PSA deficiency due to ST8Sia-II depletion does not affect the frequency of cells undergoing DNA replication in neurogenesis.

Altered Axonal Targeting and Ectopic Synapse Formation

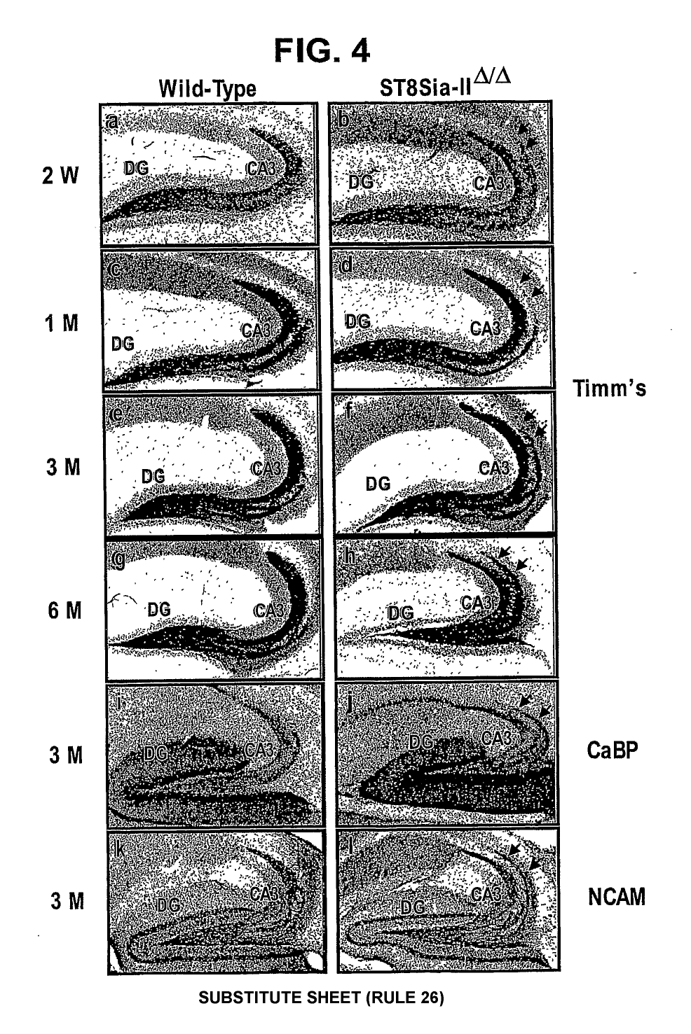

Hippocampal mossy fibers arise from granule cells in the dentate gyrus and target to the hippocampal CA3 region, fasculating to become a thick suprapyramidal fiber projection. In addition, sprouting fibers from the dentate gyrus normally form a much thinner infrapyramidal mossy fiber projection along the border between the pyramidal cell layer and stratum oriens. This infrapyramidal projection is usually shorter than the suprapyramidal mossy fiber projection and terminates or merges with the suprapyramidal projection around CA3c. However, in the ST8Sia-II deficient mice, the suprapyramidal mossy fiber projection tended to be thinner and the infrapyramidal projection was thicker. Moreover, the infrapyramidal mossy fibers often extended to the far CA3a region (FIG. 4). This aberration was already present at the age of 2 weeks, and was prominent in all older ages examined. The same pattern was found by immunostaining of NCAM protein and calbindin expression (FIG. 4). Timm's staining further revealed many fine mossy fibers invading pyramidal cell layers in the CA3 region in ST8Sia-II deficient mice, forming a web-like structure between these two mossy fiber projections (CA3b and c). Visualization of cellular features by cresyl violet staining showed that the number and the pattern of granule and pyramidal cells were not different between wild-type and mutant animals (data not shown). Thus the aberrant pattern of mossy fiber projections caused by the absence of ST8Sia-II was not due to disarrangement of pyramidal cells in the CA3 region.

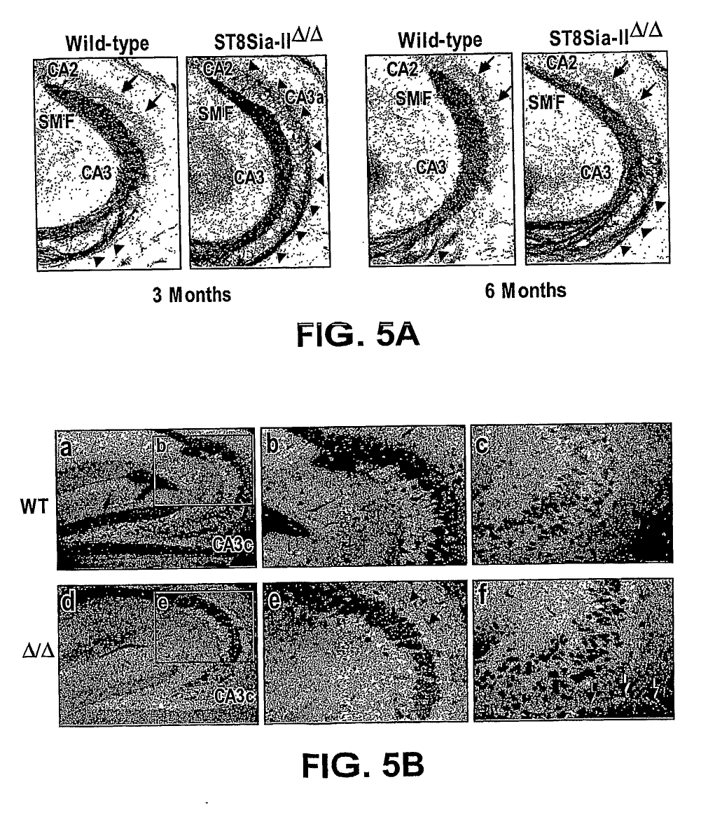

Mossy fiber axons are normally polysialylated and express NCAM, while pyramidal cells appear to express low levels of PSA. ST8Sia-IV deficient mice were previously found to lack PSA on mossy fibers but expressed PSA on neural precursors in the dentate gyrus at the age of 6 weeks (Eckhardt et al., J. Neurosci. (2000) 20:5234-5244.). In ST8Sia-II deficient mice, both suprapyramidal and infrapyramidal mossy fibers express PSA like their wild-type littermates (FIG. 5a). Suprapyramidal mossy fibers normally form synapses in the stratum oriens of CA3a. In contrast, the infrapyramidal mossy fibers normally synapse at CA3c and typically do not form synapses in the stratum oriens of CA3a. In the ST8Sia-II deficient mice, the infrapyramidal mossy fibers target into the stratum oriens of CA3a area and form ectopic synapses (FIG. 5b). In addition, mossy fibers extending along the ventral side of granule cell layer are often observed in the ST8Sia-II deficient mice and these also form ectopic synapses. These findings demonstrate alterations in neuronal circuitry in the hippocampus due to the absence of ST8Sia-II.

Hippocampal Synaptic Plasticity

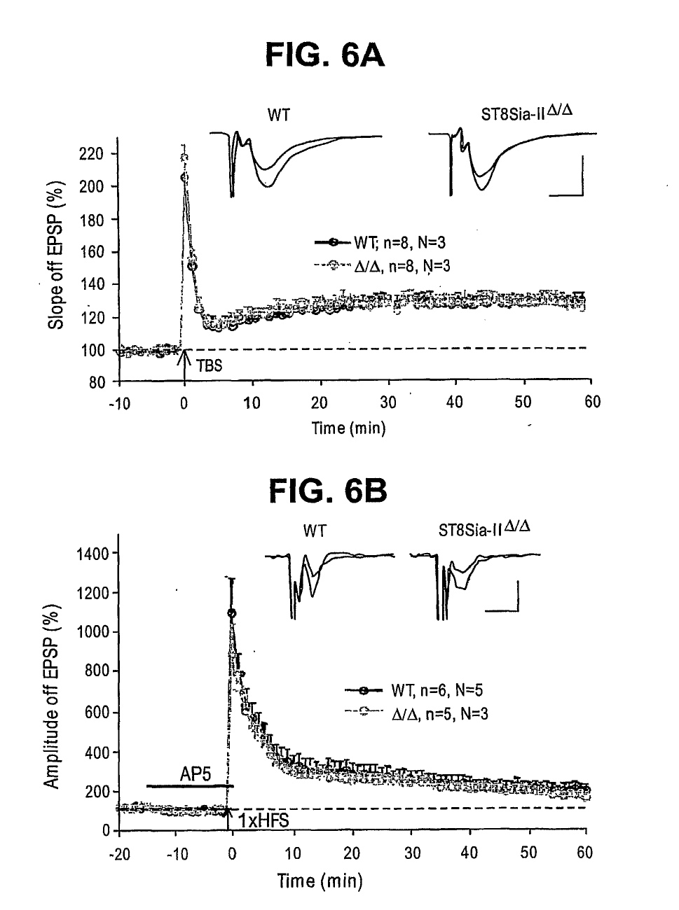

The most widely studied form of plasticity, LTP in the CA1 region of the hippocampus, has been reported to be abnormal in NCAM and ST8Sia-IV deficient mice (Muller et al., Neuron (1996) 17:413-422; Eckhardt et al., 2000, supra). Moreover, removal of PSA from hippocampal cultures of wild-type mice by endo-N prevented both LTP and LTD (Becker et al., J. Neurosci. Res. (1996) 45:143-152; Muller et al., 1996, supra). In our studies, theta-burst stimulation (TBS) of Schaffer collaterals reliably produced short-term potentiation (STP) and LTP in all slices measured from wild-type animals (FIG. 6a). The mean level of STP measured as maximal potentiation during 1 min after TBS was 216.0±10.7% and the level of LTP seen 50-60 min after TBS was 129.3±14.3% (number of slices n=8, number of mice N=3). The levels of STP and LTP in ST8Sia-II deficient mice (218.2±17.6% and 127.9±5.3%, respectively; n=8, N=3) were not significantly different from wild-type mice. Stimulus-response curves for fEPSPs evoked by stimulation of Schaffer collaterals and the mean amplitudes of responses being 50% of the supramaximal levels were not different between ST8Sia-II deficient mice and wild-type littermates, thus demonstrating normal basal levels of excitatory transmission (data not shown).

LTP at mossy fiber synapses with CA3 pyramidal cell has been reported to be abnormal in NCAM deficient mice (Cremer et al., Proc. Natl. Acad. Sci. USA (1998) 95:13242-13247). These synapses are important for hippocampal learning and memory formation (Lisman, Neuron (1999) 22:233-242; Henze et al., Neuroscience (2000) 98:407-427). CA3 LTP has features clearly distinct from CA1 LTP, being independent of postsynaptic NMDA receptors, but mediated by an elevation in cyclic adenosine monophosphate (cAMP) and activation of adenylate cyclase and PKA (Weisskopf et al., Science (1994) 265:1878-1882). Field EPSPs evoked in CA3 pyramidal cells by mossy fiber stimulation were identified using a number of criteria (see Materials and methods) and showed properties typical for mossy fiber responses: Low frequency stimulation (0.33 Hz) potentiated fEPSPs to approximately 250% in wild-type and ST8Sia-II deficient mice. Application of L-CCG1 reduced the amplitude of fEPSPs similarly in both genotypes by 80%. The NMDA receptor antagonist AP-5 did not affect the amplitude of recorded fEPSPs in either in wild-type or ST8Sia-II deficient samples.

A single train of high-frequency stimulation (1×HFS, applied in the presence of AP 5) induced robust PTP and LTP in slices from wild-type mice (1133.1±180.4% and 195.8±133.3%, respectively; n=6, N=5). The levels of PTP and LTP in ST8Sia-II deficient mice were 912.3±135.8% and 181.6±13.1%, respectively (n=5, N=3) (FIG. 6b). This was not significantly different from values among wild-type littermates. Recorded profiles of CA3 LTP resembled those observed in our previous studies (Eckhardt et al., 2000, supra; Evers et al., J. Neurosci. (2002) 22:7177-7194) and by other groups (Maccaferri et al., Science (1998) 279:1368-1370; Yeckel et al., 1999). PTP induced by a stronger induction protocol (4×HFS) was 1239.0184.3% (n=7, N=5) in wild-type and 1333.51192.5% (n=6, N=4) in ST8Sia-II deficient mice. The values measured 50-60 minutes after induction of LTP in wild-type and ST8Sia-II deficient mice were 219.2124.1% (n=7, N=5) and 257.2132.7% (n=6, N=4), respectively (data not shown). Thus, NMDA receptor-independent LTP in mossy fiber-CA3 synapses is not affected by ST8Sia-II deficiency.

Reduced Fear Conditioning Responses and Increased Exploratory Behavior

ST8Sia-II deficient mice for physical, metabolic, and behavioral abnormalities, including assays of locomotor activity, muscle strength, heart rate, blood pressure, pulmonary function, nociception, and other parameters were examined as presented in Table 1. There were no statistically significant differences between wild-type and ST8Sia-II deficient mice in these parameters. There were however consistent differences in exploratory activity, passive avoidance, and fear conditioning tasks that were replicated in two separate cohorts of mice.

TABLE 1
Assessments of basic appearance, function and metabolic parameters

| | wt/wt | | Δ/Δ | |
|---|---|---|---|---|
| Test/Assay | Mean (SEM) | | Mean (SEM) | |
| Gross Physical Assessment: | | | | |
| General appearance | Normal | | Normal | |
| Sensorimotor reflexes | Normal | | Normal | |
| Postural reflexes | Normal | | Normal | |
| Motor activity: | | | | |
| Rotarod (latency to fall-sec) | 35.7 | (10.4) | 27.5 | (2.6) |
| Initiation of movement (sec) | 13.2 | (3.8) | 16.2 | (3.1) |
| Wire hang (latency to fall-sec) | 28.6 | (4.5) | 30.1 | (3.1) |
| Grip strength (force in grams) | 373.8 | (65.6) | 393.2 | (71.8) |
| Cage-top hang test (latency to fall-sec) | 38.4 | (7.1) | 23.6 | (7.3) |
| Pole test (score) | 5.3 | (0.6) | 5.1 | (0.6) |
| Nociception | | | | |
| Hot plate (sec) | 10.6 | (1.23) | 12.1 | (1.9) |
| Tail flick (sec) | 3.0 | (0.17) | 3.5 | (0.52) |
| Blood pressure: | | | | |
| Mean (dbp + ⅓(spb − dbp)) | 95.7 | (4.4) | 90.0 | (5.2) |
| Systolic (sbp) | 144.6 | (6.5) | 140.3 | (9.2) |
| Diastolic (dbp) | 72.3 | (4.8) | 67.3 | (4.1) |
| Heart rate (bpm) | 680 | (10.9) | 655 | (14.3) |
| Pulmonary function-CO uptake | 27.9 | (1.2) | 25.5 | (1.3) |
| (microliter/min) | | | | |
| Respiration rate (bpm) | 239 | (11.4) | 231 | (11.0) |

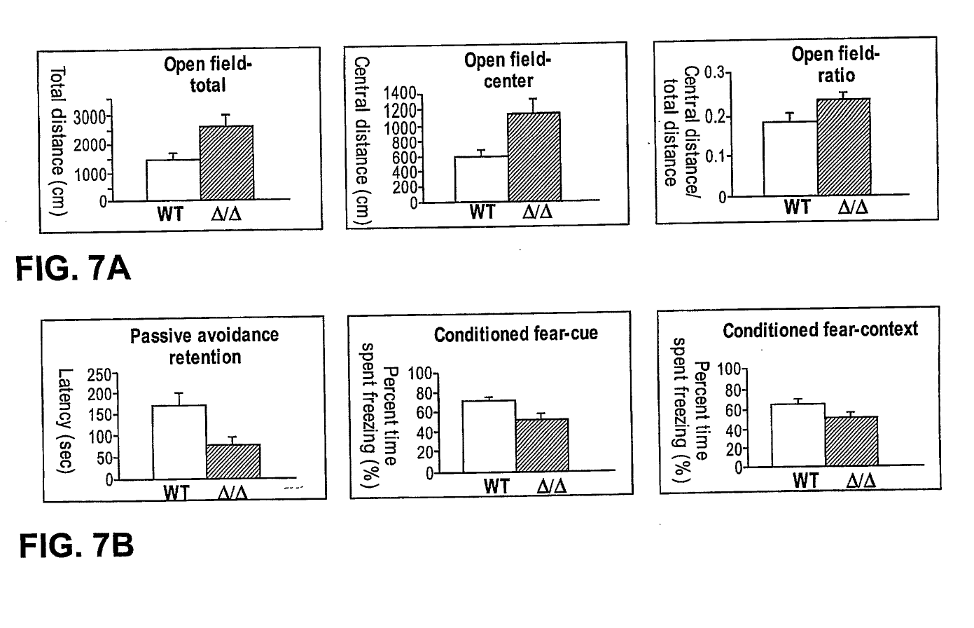

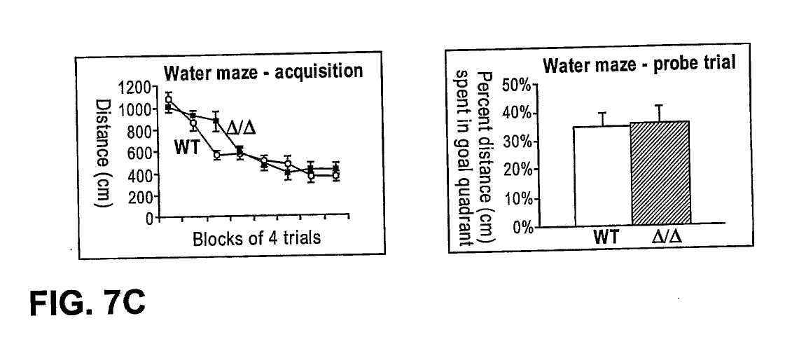

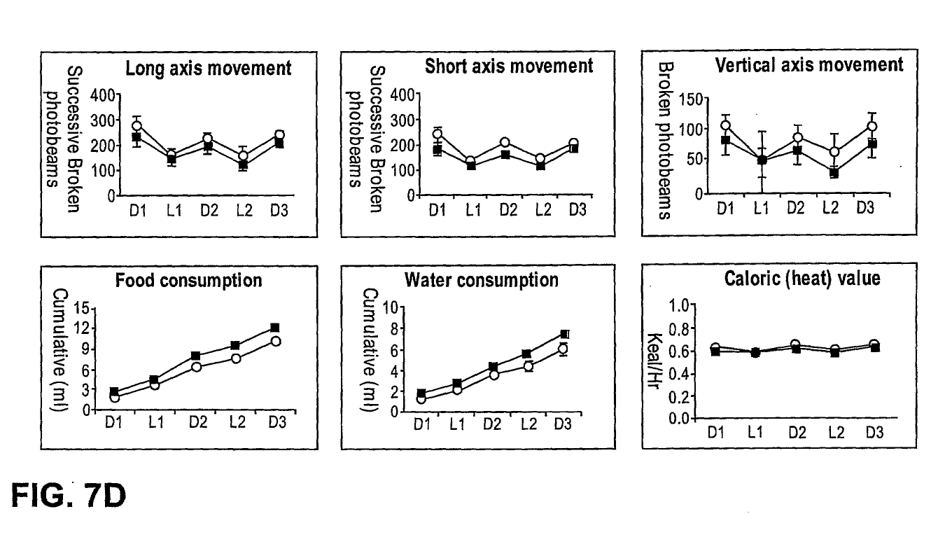

The open field test measures exploration and anxiety behaviors in a novel environment. Mice are typically anxious in this context and tend to avoid the center of a large arena. The distance traveled in the center of the arena normalized for the total distance traveled is a measure of anxiety. ST8Sia-II deficient mice traveled more total distance in the open field (t_{[48]}=2.65, p<0.01) and more distance in the center of the arena (t_{[48]}=2.9, p<0.005) (FIG. 7a). Normalizing the distance traveled in the center to the total distance traveled revealed a trend (p<0.08) of increased proportional time in the center, suggesting that ST8Sia-II deficient mice do not avoid the normally less preferred center area of the open field arena. In addition, increased rearing behavior, another indicator of exploratory activity, was observed among the second cohort of ST8Sia-II deficient mice tested with the mean number of upper level photo-beams broken measured at 188±16 for wild-type mice and 313±42 for the ST8Sia-II deficient littermates (t_{[1,19]}=3.0, p<0.01).

In the passive avoidance task, the ST8Sia-II deficient mice were significantly impaired (t_{[47]}=2.79, p<0.007), failing to increase their latency to enter the dark chamber on the second day (FIG. 7b). There was no difference in latency to enter the dark chamber on the training day (wild-type: 40±14; Δ/Δ: 36±9).

Behavioral responses to two standard fear-conditioning tasks were also abnormal in ST8Sia-II deficient mice. The association of a single cue (i.e. a tone) with an unpleasant electrical shock is considered unimodal and critically depends upon neural processing in the amygdala but not hippocampus. In contrast, associating context (the spatial and environmental cues making up the test chamber) with the unpleasant stimulus is considered multimodal and requires neural processing in both amygdala and hippocampus (Fanselow and Le Doux, Neuron (1999) 23:229-232). ST8Sia-II deficient mice were significantly impaired in both the cued (t_{[48]}=2.56, p<0.01) and contextual (t_{[48]}=2.23, p<0.05) versions of the fear conditioning test (FIG. 7b). ST8Sia-II deficient mice had a normal nociception response (Table 1) and were not impaired in the acoustic startle test (FIG. 7c), indicating that the deficit in fear conditioning to cue was not due to an inability to experience discomfort or to hear the tone.

ST8Sia-II deficient mice were also analyzed for spatial learning in the water maze test. NCAM-deficient mice exhibited a defect in this task, consistent with the finding of a defect in LTP in synapses formed by the Schaffer collaterals in the hippocampal CA1 region (Cremer et al., Nature (1994) 367:455-459; Muller et al., Neuron (1996) 17:413-422). There was however no significant difference between the ST8Sia-II deficient and wild-type mice in the water maze acquisition trials or in probe trial performance (FIG. 7d), suggesting that spatial learning and associated memory are not altered by ST8Sia-II deficiency. This is also in accordance with the results of the electrophysiological measurements that showed normal LTP of hippocampal CA1 (and CA3) in the ST8Sia II deficient mice.

Performance of passive avoidance and fear conditioning tasks requires that the animals are able to control their movement. It was possible that if ST8Sia-II deficient mice were inherently hyperactive, and thereby would perform poorly without bearing impairments specifically in fear memory. For this reason, we quantitatively analyzed motor activity and basic physiologic parameters over multiple light and dark cycles using automated metabolic chambers. It is noteworthy that ST8Sia-II deficient mice did not show an increase in motor activity and other parameters assessed, and continued to exhibit circadian rhythm (FIG. 7e). These results support the view that altered fear conditioning and exploratory behavior occurring in ST8Sia-II deficiency are due to alterations in processing and accessing the stimuli leading to provoking anxiety and fear memory.

It is widely accepted that the amygdala is involved in fear memory (LeDoux, Annu. Rev. Neurosci. (2000) 23:155-184; Maren, Annu. Rev. Neurosci. (2001) 24:897-931). Since PSA is expressed in the amygdala (Nacher, Neuroscience (2002) 113:479-484), we investigated possible PSA alterations in ST8Sia-II deficiency. Similar PSA expression was detected in the amygdala, hypothalamus, and piriform cortex of adult ST8Sia-II deficient mice and wildtype littermates (data not shown), indicating that PSA deficiency does not occur in this ST8Sia-II deficient model of defective fear conditioning. Furthermore, retrograde labeling with BDA injected into the amygdala demonstrated a normal trace pattern in the hippocampus of ST8Sia-II deficient mice (data not shown), as compared with previous findings (Pikkarainen et al., J. Comp. Neurol. (1999) 403:229-260.; Berretta et al., J. Comp. Neurol. (2001) 431:129-138; Petrovich et al., Brain Res. Rev. (2001) 38:247-289.).

Discussion

Differential Expression and Activity of ST8Sia-II and ST8Sia-IV polysialyltransferases

The ST8Sia-II polysialyltransferase is required for the formation of a subset of the total PSA biosynthetic repertoire expressed in the brain. We further observed that ST8Sia-II forms PSA in a spatial and temporal pattern that functions distinctly from PSA produced by ST8Sia-IV. We also noted partial overlap with the NCAM deficient phenotype, suggesting that the functions of NCAM are in some contexts dependent upon PSA modification by ST8Sia-II. Deficiency of ST8Sia-II caused an abnormality in the targeting of infrapyramidal hippocampal mossy fibers resulting in ectopic synapse formation in CA3.

Neural cell adhesion, migration, axonal growth and neuronal plasticity have been clearly attributed to PSA, although the relative contribution of ST8Sia-II, ST8Sia-IV, and NCAM to these events is not completely defined. During development, ST8Sia-II contributes to the majority of PSA formation in specific brain regions, including granule cells of the dentate gyrus and neural precursor cells in the SVZ. However, PSA deficiency by ST8Sia-II inactivation does not alter mitotic events in neurogenesis. In neuronal differentiation, PSA remains expressed on mossy fiber axons and migrating cells in the RMS or striatum, most likely due to the activity of ST8Sia-IV. In fact, adult mice lacking ST8Sia-IV are highly deficient in PSA among multiple brain compartments including the mossy fibers and striatum (Eckhardt et al., 2000, supra). These results indicate different contributions by ST8Sia-II and ST8Sia-IV to PSA formation involving temporal and likely cell-type-specificities that promote the unique functions that can now be ascribed to these two polysialyltransferases.

Migrating cells in the RMS express PSA in the absence of either ST8Sia-II or ST8Sia-IV suggesting that in some contexts the functions of both polysialyltransferases may also overlap. Tangential migration from the SVZ to the olfactory bulb is a relatively long distance, and both polysialyltransferases may operate in this migration. In contrast, even though granule cells express both ST8Sia-II and ST8Sia-IV transcripts (Angata et al., J. Biol. Chem. (1997) 272:7182-7190; Hildebrandt et al., J. Neurochem. (1998) 71:2339-2348), they appear to express PSA initially by ST8Sia-II, but subsequently by ST8Sia-IV when they extend mossy fiber axons. It is therefore also possible that PSA synthesis may be further regulated at the level of polysialyltransferase RNA translation.

Distinct and Overlapping Functions of Polysialyltransferases and NCAM

From characterization of mice deficient in NCAM, ST8Sia-II, or ST8Sia-IV, we can compare the biological roles of PSA and NCAM (Table 2). NCAM-deficient mice (intact ST8Sia-II and ST8Sia-IV expression), and mice treated with endo-N to remove PSA, have a thicker RMS and a smaller olfactory bulb, perhaps due to slower tangential migration of precursor cells (Hu et al., Neuron (1996) 16:735-743). In contrast, mice lacking either ST8Sia-II or ST8Sia-IV have a normal RMS and olfactory bulb, suggesting that PSA produced by either ST8Sia-II or ST8Sia-IV is sufficient for olfactory precursor cells to migrate properly, or that additional polysialyltransferases operate in the synthesis of PSA in these cells.

TABLE 2
Comparison of polysialic acid and NCAM function
in neural development and synaptic plasticity

| | | ST8Sia-IV | NCAM | ST8Sia-II | |
|---|---|---|---|---|---|
| Function | Endo-N | deficiency | deficiency | deficiency | Basis of Defect |
| Migration of | − | + | − | + | PSA-dependent |
| neural precursors | | | | | NCAM function |
| LTP in CA1 | − | − | − | + | PSA- and ST8Sia-IV- |
| | | | | | dependent NCAM |
| | | | | | function |
| LTP in CA3 | + | + | − | + | PSA-independent |
| | | | | | NCAM function |
| Lamination of | − | + | − | − | PSA- and ST8Sia-II- |
| mossy fibers | | | | | dependent NCAM |
| | | | | | function |

All studied forms of short- and long-term hippocampal synaptic plasticity appeared to be normal in adult ST8Sia-II deficient mice. In contrast, adult ST8Sia IV-deficient mice are deficient in LTP and LTD in the hippocampal CA1 region (Eckhardt et al., 2000, supra). This is consistent with low levels of ST8Sia-II expression in adulthood and the continued expression of ST8Sia-IV in the hippocampus of adult ST8Sia-II deficient mice. These studies underscore the unique role of ST8Sia-IV in the formation of CA1 LTP in adult mice. NMDA receptor-independent LTP in hippocampal CA3 pyramidal cells was abolished in NCAM-deficient mice (Cremer et al., Proc. Natl. Acad. Sci. USA (1998) 95:13242-13247). This contrasts with findings of normal CA3 LTP in adult mice lacking either ST8Sia-II or ST8Sia-IV, or ST8Sia-IV deficient mice treated with endo-N (Eckhardt et al., 2000, supra). Taken together these findings support the view that the NCAM protein backbone is required for LTP in the CA3 region of the adult hippocampus.

Polysialic Acid in Axon Targeting and Synapse Formation

When axons are misguided and do not meet the proper target cells, they are typically retracted and eventually die (Tessier-Lavigne and Goodman, Science (1996) 274:1123-1133; Pettmann and Henderson, Neuron (1998) 20:633-647; Dickson, Science (2002) 298:1959-1964). The hippocampal mossy fiber pathway from the granule cells of the dentate gyrus to the pyramidal cells of CA3 is a particularly interesting system for studying these processes. Neural precursor cells arise in the most inner layer of the dentate gyrus, and subsequently migrate into the granule cell layer, where they differentiate into granular neurons that extend dendrites and axons (Seki and Arai, Neurosci. Res. (1993) 17:265-290; Gage, Science (2000) 287:1433-1438). The suprapyramidal bundle of mossy fiber axons, which also express PSA, target towards CA3 pyramidal cells to form synapses. The infrapyramidal mossy fibers, the secondary blade of mossy fiber axons, initially project into the area below CA3 pyramidal cells and merge with suprapyramidal mossy fibers after crossing through the pyramidal cell layer (Gaarslkjaer, Brain Res. (1986) 396:335-357).

We observed abnormal axonal targeting of the infrapyramidal mossy fiber projection in ST8Sia-II deficient mice, and although these axons continued to express PSA, they formed ectopic synapses in the CA3a region. NCAM180-deficient mice exhibit a similar altered morphology of infrapyramidal mossy fiber projection (Seki and Rutishauser, J. Neurosci. (1998) 18:3757-3766). In contrast, this phenotype was not observed among ST8Sia-IV deficient mice, indicating that correct targeting of infrapyramidal mossy fiber projections involves a function of PSA that is dependent upon ST8Sia-II and NCAM (Table 2).

ST8Sia-II and PSA in Fear and Anxiety Behaviors

Infrapyramidal mossy fiber projections vary among different mouse and rat strains and appear to correlate with reduced anxiety, increased exploration, and reduced responses to fear conditioning (van Daal et al., Behav. Brain Res. (1991) 43:57-64; Henze et al., Neuroscience (2000) 98:407-427). In the open field test, inbred mouse strains with larger intra- and infrapyramidal mossy fiber fascicles habituate faster to a novel environment and have reduced fear behavior (Crusio, Behav. Brain Res. (2001) 125:127-132). Additionally, mice engineered with a defective Dcx gene, a cause of classical type 1 lissencephaly in human, have a similar defect in mossy fiber projection and exhibit reduced freezing to conditioned fear assays in both cue- and context-associated tasks (Corbo et al., J. Neurosci. (2002) 22:7548-7557). In rats, the DA rat strain exhibits significantly larger infrapyramidal mossy fibers with reduced freezing and high rearing activities, whereas BDE rats with shorter infrapyramidal mossy fibers show freezing and low rearing activity (Prior et al., Behav. Brain Res. (1997) 87:183-194). These behavioral changes are similar in CHL1 mutant mice that also have larger intra- and infrapyramidal mossy fiber projections (Montag-Sallaz et al., Mol. Cell. Biol. (2002) 22:7967-7981).

ST8Sia-II deficiency alters fear responses normally processed by both the hippocampus and amygdala. Although ST8Sia-IV deficient mice have not been similarly studied, NCAM deficiency leads to the behavioral changes detected in ST8Sia-II deficiency (Stork et al., Eur. J. Neurosci. (2000) 12:3291-3306). In contrast to ST8Sia-II deficiency, NCAM deficiency also resulted in impaired spatial learning memory, which is consistent with the impairment of LTP in Schaffer collaterals of the CA1 region (Cremer et al., Nature (1994) 367:455-459; Muller et al., 1996, supra).

Genetic control of ST8Sia-II and ST8Sia-IV expression appears to underlie the distinct neurological processes mediated by PSA function. PSA can play different roles in axonal projections that may also reflect the strength of axon-axon and axon-environment interactions (Marx et al., Development (2001) 128:4949-4958). In the adult hippocampus, chronic stress as well as treatment with glucocorticoid stress hormones alters neural plasticity involving mossy fiber terminals and CA3 pyramidal cells, which may reflect the associated changes in hippocampal PSA and NCAM expression (Rodriguez et al., Eur. J. Neurosci. (1998) 10:2994-3006; Sandi et al., Neuroscience (2001) 102:329-339; Sandi, et al., Biol. Psychiatry (2003) 54:599-607). Furthermore, kindling of the amygdala, but not the entorhinal cortex, induces sprouting of infrapyramidal mossy fibers in the CA3 region (Represa et al., Neurosci. Lett. (1989) 99:345-50).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of ameliorating an anxiety response in a mammal in need thereof, the method comprising administering to the mammal a compound that inhibits ST8Sia-II sialyltransferase activity.

2. The method of claim 1, wherein the compound comprises an ST8Sia-II sialyltransferase substrate analog.

3. The method of claim 2, wherein said substrate analog is an analog of a donor substrate.

4. The method of claim 3, wherein said analog is a competitive inhibitor of said donor substrate.

5. The method of claim 3, wherein said analog is a noncompetitive inhibitor of said donor substrate.

6. The method of claim 3, wherein said analog of a donor substrate is an analog of CMP-sialic acid.

7. The method of claim 2, wherein said substrate analog is an analog of an acceptor substrate.

8. The method of claim 7, wherein said analog is a competitive inhibitor of said acceptor substrate.

9. The method of claim 7, wherein said analog is a noncompetitive inhibitor of said acceptor substrate.

10. The method of claim 7, wherein said analog of an acceptor substrate is an analog of a sialic acid selected from the group consisting of an α-2,3-linked sialic acid, an α-2,6-sialic acid and an α-2,8-linked sialic acid.

11. The method of claim 1, wherein said mammal is a human.

12. The method of claim 1, wherein said anxiety response comprises fear.

13. The method of claim 1, wherein said anxiety response comprises depression.

14. A method of identifying a compound for use in inhibiting an anxiety response in a mammal, the method comprising:
a) providing an assay mixture which comprises: an ST8Sia-II sialyltransferase, a potential anxiety response modulator, a cytidine 5′-monophosphate (CMP)-sialic acid donor saccharide, an acceptor saccharide, and additional reagents required for ST8Sia-II sialyltransferase activity;
b) incubating the assay mixture under conditions in which the ST8Sia-II sialyltransferase is active; and
c) determining whether the amount of sialic acid transferred to the acceptor saccharide is increased or decreased in comparison to an assay mixture which lacks the potential anxiety response modulator;
wherein a potential anxiety response modulator which results in a decrease in sialic acid transfer to the acceptor saccharide is suitable for inhibiting an anxiety response.

15. The method of claim 14, wherein said acceptor saccharide for the ST8Sia-II sialyltransferase comprises an asparagine (N-) linked glycan.

16. The method of claim 15, wherein the N-linked glycan comprises at least one terminal sialic acid moiety selected from the group consisting of an α2-3-linked terminal sialic acid, an α2-6-linked terminal sialic acid, and an α2-8-linked terminal sialic acid.

17. A method of identifying compounds for inhibiting an anxiety response in a mammal, the method comprising:
providing a cell which comprises a polynucleotide that encodes a ST8Sia-II polysialyltransferase, an acceptor saccharide for the ST8Sia-II sialyltransferase, and CMP-sialic acid;
contacting the cell with a potential anxiety response modulator and incubating the cell under conditions in which the ST8Sia-II sialyltransferase is normally expressed; and
determining whether the polysialic acid (PSA) level is increased or decreased compared to the PSA level in the absence of the potential anxiety response modulator;
wherein a potential anxiety response modulator that causes a decrease in the amount of PSA produced is useful for inhibiting an anxiety response in a mammal.

18. The method of claim 17, wherein said acceptor saccharide for the ST8Sia-II sialyltransferase is an asparagine (N-) linked glycan.

19. The method of claim 18, wherein the N-linked glycan comprises at least one terminal sialic acid moiety selected from the group consisting of an α2-3-linked terminal sialic acid, an α2-6-linked terminal sialic acid, and an α2-8-linked terminal sialic acid.
